(19) United States
(12) Patent Application Publication
(10) Pub. No.: US 2023/0197228 A1
(43) Pub. Date: Jun. 22, 2023

(54) ANESTHESIOLOGICAL PROCEDURE RECORDING SYSTEM AND METHOD

(71) Applicants: Roberto Eduardo Orofino Giambastiani, La Plata (AR); Rodrigo Mariano Sáenz, La Plata (AR); Guido Lahitte Battistoni, City Bell (AR); Juan Ignacio Umaran, City Bell (AR)

(72) Inventors: Roberto Eduardo Orofino Giambastiani, La Plata (AR); Rodrigo Mariano Sáenz, La Plata (AR); Guido Lahitte Battistoni, City Bell (AR); Juan Ignacio Umaran, City Bell (AR)

(21) Appl. No.: 17/553,037

(22) Filed: Dec. 16, 2021

(51) Int. Cl. G16H 20/10; G16H 10/60; G16H 40/63; G01S 5/02

(52) U.S. Cl. CPC G16H 20/10; G16H 10/60; G16H 40/63; G01S 5/0295

(57) ABSTRACT

A system and method for recording an anesthesiological procedure is provided. The system is configured to capture physiological data of a patient and of their interaction with electromechanical devices to which the patient is connected and data about the temporal-spatial relationship between an anesthesiologist and the patient; and transmit the collected data in real time to a remote storage system wherein an immutable copy of such data is stored, so that the anesthesiological procedure can be fully reconstructed later.

### FIELD OF INVENTION

The present invention is framed within the field of medicine and anesthesiology in particular. It is mainly applicable to procedures that require active and continuous technological monitoring of a patient by a health professional such as an anesthesiologist.

The term “technological monitoring” refers to the use of technology to monitor the vital parameters of patients and their interaction with the medical instruments and equipment connected to them. In contrast, “non-technological monitoring” is based solely on the observation of clinical parameters of patients.

Whereas the term “active monitoring” refers to the observation and interpretation of signals by a health professional. Conversely, “passive monitoring” does not require observation or interpretation of signals and may be performed by setting automatic alarms.

Finally, the term “continuous monitoring” refers to the constant monitoring of a patient by a health professional. In contrast, “discontinuous monitoring” is performed without the constant presence of a medical doctor, who may instead execute an intermittent monitoring of the patient and even monitor several patients simultaneously.

Furthermore, due to its characteristics, this invention is directed fundamentally to intraoperative anesthesiological procedures, including surgical procedures and technical procedures which are implemented, for instance, in hemodynamics, gastroenterology and imaging service rooms.

### BACKGROUND

So as to facilitate the understanding of the role of an anesthesiologist in intraoperative anesthesiological procedures, a brief summary of theory of control will be provided below.

Control theory mainly studies the behavior of dynamical systems and how to make them behave in the desired way. A dynamical system is capable of receiving stimuli or excitations (inputs) and of exhibiting, due to these inputs, responses (outputs). Whereas a control system is a type of system that allows influencing the operation of the dynamical system. The purpose of a control system is to achieve, via the manipulation of control variables, governance over the output variables, so that they may reach predetermined values (setpoints). The basic components of a control system are: one or more sensors that measure the values of the output variables of the system; one or more controllers that use the values measured by the sensors and the control setpoint (reference) to determine the action to be implemented so as to modify the control variables; and one or more actuators that execute the corrective action determined by the controller via modifying the control variables.

By analogy, the dynamical system may represent a patient who undergoes an anesthesiological procedure and the controller may represent an anesthesiologist who acts on the patient and the equipment connected to them. The duty of the anesthesiologist is to interpret signals and modify variables so as to prevent physiological and biochemical parameters from deviating from the normal ranges for each situation.

Additionally, the control system may include automatic controllers for actuators such as drug infusion pumps and mechanical ventilators. In such cases, the anesthesiologist also fulfills the role of a supervisor of those automatic controllers.

So as to perform their tasks, the anesthesiologist directly observes the patient and uses monitoring equipment (including those of the anesthesia machine) capable of capturing and displaying physiological data from the patient, as well as data on the interaction between the patient and the electromechanical equipment connected to them. By analyzing the displayed data and comparing it with the reference values, the anesthesiologist makes decisions about the actions to be implemented on the patient and on the electromechanical equipment connected to them to keep the patient safe throughout the anesthesiological procedure and until they regain their own control or it is exerted by another controller (such as an intensive care service).

In this context, “maintaining patient safety” implies maintaining their physiological and biochemical parameters within limits determined by scientific consensus as normal according to each specific situation, and acting efficiently to restore them when they deviate from such limits. The lack of timely correction of deviations in physiological or biochemical parameters may generate potential harm to the patient. Such harm may be reversible, irreversible (causing potential sequelae) or even cause the death of the patient. Any event that causes deviations from such parameters and threatens the integrity of the patient is considered a “harm”.

The concept of “harm” is very broad and includes from general events such as hypoxia, pain, acidosis and hypothermia, to more limited concepts such as burns, cuts and compressions. The role of the controller is to efficiently prevent harm. Failure in harm prevention is considered suboptimal performance.

Suboptimal performance of a controller may be framed in four main failures:
Detection failure: the controller does not detect that an abnormality or irregularity is occurring.
Diagnostic failure: the controller detects that an abnormality or irregularity is occurring, but misidentifies the situation.
Therapy selection failure: the controller detects that an abnormality or irregularity is occurring, identifies the situation, but misinterprets the treatment to be applied.
Failure in therapy implementation: the controller detects that an abnormality or irregularity is occurring, identifies the situation, identifies the correct treatment, but executes it incorrectly or ineffectively.

These main failures may be consequence of:
Ineffective surveillance (absence, distraction).
Insufficient knowledge.
Insufficient training.
Ineffective supervision (of students/residents by the professional in charge).
Insufficient resource availability.
Resources malfunction.

Anesthesiology is a medical specialty that employs measurements, collection, and documentation of data about the course of surgical or technical procedures. Physiological and biochemical measurements as well as manipulations and configurations of the technological devices employed on the patient are included here. This data becomes the basis for decision-making processes, so the quality of the record may influence the protocols executed and, therefore, the performance of the anesthesiologist.

A main reason for recording anesthesiological procedures is that such evidence may serve as support for future actions taken by other anesthesiologists or health providers in similar scenarios, that is, to promote decision-making based on scientific information. Moreover, records of anesthesiological procedures may serve as evidence in court proceedings.

Over time, various techniques for recording anesthesiological data have been adopted.

The most elementary recording systems consist of paper record sheets that the anesthesiologist manually completes on a regular basis, transcribing data and interpretations from the monitors. For years, this was the main documentation method.

More complex recording systems are usually automatic and integrated into the anesthesia machines or monitors. The data is stored centrally, either in the internal memory of a monitor, or in a server belonging to a hospital to which the monitors are connected via a local network.

There are also hybrid documentation methodologies in which part of the data is recorded automatically and the other is collected manually.

Known anesthetic recording methodologies may be considered suboptimal from various aspects.

Non-automatic anesthesia record systems, such as paper record sheets, present numerous disadvantages. By assigning the professional the task of transcribing measurements, it distracts them from their role as supervisor and controller. Furthermore, such transcripts are susceptible to errors, omissions, do not provide continuous sampling of data, and do not guarantee fidelity in the measurement record.

Other disadvantages of paper documentation include recall bias, since transcripts often occur after an event has taken place or even after the procedure, and also the inclusion of illegible information.

Paper record sheets are also susceptible to content adulteration, forgery, destruction and accessibility limitation. Additionally, they present a precarious way of authenticating the person who generated the content, certifying precise time schedule, determining procedure geolocation, and protecting patient privacy.

Automatic recording systems allow data to be recorded in real time, freeing anesthesiologists from having to transcribe it manually, providing better readability and more accurate data capture compared to non-automatic methods. However, current automatic recording systems do not possess professional authentication mechanisms other than those for controlling access to a monitor or anesthesia machine by password, they do not provide GPS certified geolocation or UTC synchronized schedules. They are also based on centralized storage which is susceptible to adulterations and access limitations.

Data integrity and accuracy should be guaranteed in every process from sampling, transmission, storage and recovery in order to be useful for patient care, data analysis and audit of medical practice.

Records from current systems, whether automatic or manual, may be susceptible to damage, modification or destruction that may impair the record of truth.

A complete record of the performance of a control system should include data of the relation between their components: controlled unit and controller unit. In this way, current anesthesia record systems do not provide complete data in order to fully reconstruct what takes place during anesthesia procedures as they do not provide data of spatio temporal relation between patients and their anesthesiologists.

A suboptimal record reconstruction may be caused by:
Under-recording.
Malfunction of the recording system.
Adulteration of the record.
Destruction of the record.
Obstruction to the accessibility of the record.

Existing solutions do not provide immutable nor complete records for performance analysis. Systems should allow transparent reconstruction and interpretation of events to prompt quality improvement and patient safety.

Consequently, there is a need for efficient systems and methods for recording data that promote the generation of high quality scientific information and that discourage acts that directly or indirectly reduce patient safety during anesthesiological procedures, such as simultaneous anesthesia, prolonged absence of the anesthesiologist in the operating room, insufficient surveillance to patient parameters, excessive latency to detect adverse events, adulteration of procedural schedules, lack of supervision of anesthesiologists in training, lack of essential medical supplies, deficient hospital equipment or inadequate maintenance.

Furthermore, anesthesia record systems should be compatible with upcoming closed loop control system technologies based on artificial intelligence in order to provide an independent and impartial sampling system which may be also used as an input data provider and performance recorder.

### SUMMARY

The invention provides in a first aspect a recording system for anesthesiological procedures comprising:

at least one first data acquisition device connectable to one or more sensors capable of capturing physiological data of a patient and of their interaction with electromechanical devices to which the patient is connected;

at least one electronic device portable by at least one anesthesiologist; and

at least one computing device connected, or integrated, to the at least one first data acquisition device and connected to the at least one portable device,

wherein the at least one portable device is configured to generate, via interaction with the at least one computing device and/or the at least one first data acquisition device, data related to the temporal-spatial relationship between the at least one anesthesiologist and the patient and, therefore, to the quality of surveillance provided by the at least one anesthesiologist to the patient, and

wherein the at least one computing device is configured to receive and transmit the collected data in real time to a remote storage system wherein an immutable copy of such data is stored.

In a second aspect of the invention, a method for recording anesthesiological procedures is provided comprising the steps of:

receiving in the at least one computing device a login credential from at least one anesthesiologist who accesses the at least one computing device and, in response to the authentication of the anesthesiologist via an authentication system:

generating at least one file to contain data on the anesthesiological procedure;

receiving in the at least one computing device preliminary data about the patient and/or about the procedure to be performed, via an input device connected to, or integrated into it, and send that preliminary data to the remote storage system;

receiving confirmation from the at least one anesthesiologist, via authentication by the at least one computing device, that the anesthesiological procedure is about to begin;

in response to receiving confirmation that the anesthesiological procedure is about to begin, start capturing physiological data by means of sensors and data related to the temporal-spatial relationship between the at least one anesthesiologist and the patient and, therefore, to the quality of surveillance provided by the at least one anesthesiologist to patient, via the interaction between the at least one portable device with the at least one computing device and/or the at least one first data acquisition device, and transmitting in real time such collected data from the at least one first data acquisition device and portable device to the at least one computing device and from there to the remote storage where an immutable copy of the collected and preliminary data linked via the at least one generated file is stored;

receiving confirmation from the at least one anesthesiologist, by authentication on the at least one computing device, that the anesthesiological procedure has ended; and

in response to receiving confirmation of completion of the anesthesiological procedure, finishing the transmission and recording of data.

### BRIEF DESCRIPTION OF DRAWINGS

The invention is described in the Detailed Description with reference to the attached drawings. The usage of the same reference numbers in different instances in the description and the figures may indicate similar or identical elements.

FIG. 1 is a schematic view of an exemplary embodiment of an anesthesiological procedure recording system in accordance with the present invention.

FIG. 2 illustrates an implementation example in which a first data acquisition device of FIG. 1 is shown in greater detail.

FIGS. 3 and 4 illustrate an implementation example in which a computing device of FIG. 1 is shown in greater detail.

FIG. 5 illustrates an implementation example in which a portable device of FIG. 1 is shown in greater detail.

FIGS. 6 to 8 illustrate a possible embodiment of the present invention, in which the anesthesiological procedure recording system additionally comprises a video synchronization device.

FIG. 9 illustrates a flowchart of a method for recording anesthesiological procedures in accordance with one embodiment of the present invention.

FIG. 10 illustrates a flowchart of a method for saving a record of an anesthesiology procedure in accordance with one embodiment of the present invention.

FIG. 11 illustrates a flowchart of a method for accessing a record of an anesthesiology procedure in accordance with one embodiment of the present invention.

FIG. 12 illustrates a flowchart of a method for adding a correction or new data to an anesthesiological procedure record in accordance with one embodiment of the present invention.

FIG. 13 illustrates a flowchart of a method for adding a correction or new data to an anesthesiological procedure record in accordance with another embodiment of the present invention.

Although the exemplary embodiments described herein are susceptible to various modifications and alternative forms, specific embodiments have been shown by way of example in the drawings and will be described in detail below. However, the exemplary embodiments described herein are not intended to be limited to the particular forms disclosed. Rather, the present disclosure covers all modifications, equivalents, and alternatives that fall within the scope of the appended claims.

### DETAILED DESCRIPTION

The general operating characteristics and advantages of the present invention will now be described in greater detail in this section in connection with the preferred embodiments, which should be considered as solely exemplary and not limiting of the present invention.

In a first aspect, the present invention is directed to a recording system for anesthesiological procedures that comprises:

at least one first data acquisition device connectable to one or more sensors capable of sampling physiological data of a patient and of their interaction with electromechanical devices to which the patient is connected;

at least one electronic device portable by at least one anesthesiologist; and

at least one computing device connected, or integrated, to the at least one first data acquisition device and connected to the at least one portable device,

wherein the at least one portable device is configured to generate, via interaction with the at least one computing device and/or the at least one first data acquisition device, data related to the temporal-spatial relationship between the at least one anesthesiologist and the patient and, therefore, to the quality of surveillance provided by the at least one anesthesiologist to the patient, and

wherein the at least one computing device is configured to receive and transmit the collected data in real time to a remote storage system wherein an immutable copy of such data is stored.

In an embodiment of the invention, the system additionally comprises at least one second data acquisition device connectable to one or more sensors capable of measuring the environmental conditions at the site where the anesthesiological procedure is executed.

Each of the devices belonging to the system of the present invention may include at least one network interface that allows communications over at least one network. The aforementioned network interface(s) may include one or more of any type of network interface (such as a wired or wireless network interface card (NIC)), an IEEE 802.11 wireless LAN (WLAN) interface, a worldwide interoperability for microwave access (Wi-MAX) interface, an Ethernet interface, a universal serial bus (USB) interface, a mobile phone network interface, a Bluetooth™ interface, a proximity data transmission interface (NFC), etc. The network may include, but is not limited to, a cellular network, a point-to-point dial-up connection, a satellite network, the Internet, a local area network (LAN), a wide area network (WAN), a personal area network (PAN), a WiFi network, an ad hoc network, or a combination thereof. The network may include one or more connected networks (e.g., a multi-network environment) which include public networks, such as the Internet, and/or private networks.

Preferably, the at least one computing device of the system is capable of identifying the quality of communications, such as the robustness of the connection based, for example, on signal quality and power, bandwidth, latency, and sending this information in real time along with the other collected data to a remote storage system, where an immutable copy of such data is stored. Any known technical way of determining the quality of a communication is applicable to this invention.

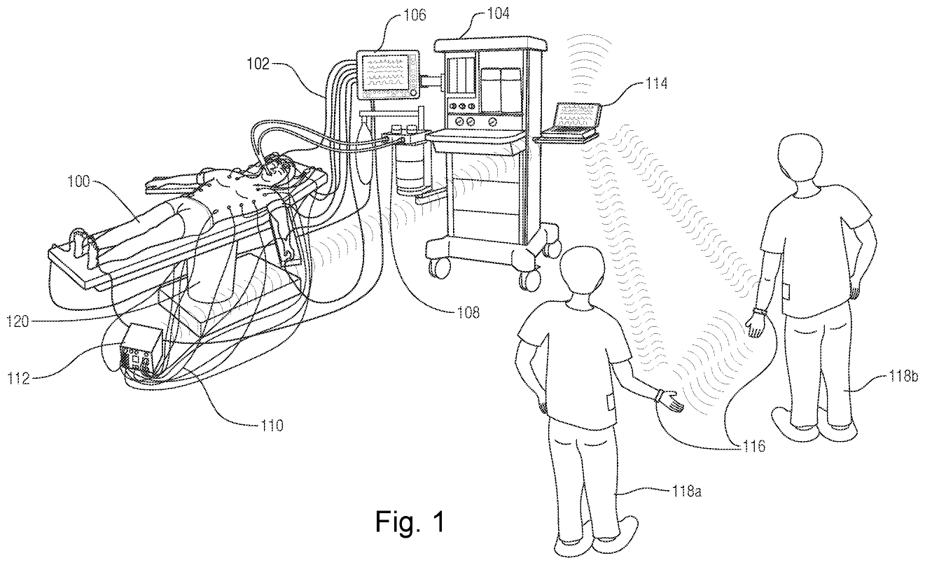

FIG. 1 shows an exemplary embodiment of an anesthesiological procedure recording system in accordance with the present disclosure. As shown in FIG. 1, a patient 100 who is to undergo an anesthesiological procedure has sensors 102 connected to an anesthesia monitor 106 integrated to an anesthesia machine 104 and, in addition, sensors 110 connected to at least one first data acquisition device 112. The at least one first data acquisition device 112 is wirelessly connected to at least one computing device 114 and, optionally, to at least one device 116 portable by at least one anesthesiologist 118a, 118b. Each portable device 116 is, in turn, wirelessly connected to the at least one computing device 114. During an anesthesiological procedure, sensors 110 capture physiological data from the patient 100 and their interaction with electromechanical devices to which the patient 100 is connected, such as a ventilation system 108 of the anesthesia machine 104 and they send the data to the at least one computing device 114. The at least one electronic device 116 portable by at least one anesthesiologist 118a, 118b interacts with the at least one computing device 114 and/or the at least one first data acquisition device 112, generating data related to the temporal-spatial relationship between the at least one anesthesiologist 118a, 118b and the patient 100 and, therefore, to the quality of surveillance provided by the at least one anesthesiologist 118a, 118b to the patient 100. The at least one computing device 114 collects all the data generated and transmits it in real time to a remote storage system, where an immutable copy of that data is stored.

The at least one first 112 and second 312 data acquisition devices may comprise at least one of: a signal conditioning circuit, a signal converter, and a bus. The signal conditioning circuit manipulates a signal in such a way that it is suitable for input to a converter. The signal conditioning may include amplification, attenuation, filtering, and isolation.

In an embodiment of the present invention, the at least one first data acquisition device 112 is wirelessly connected to the at least one computing device 114, as shown in FIG. 1. In an even more preferred embodiment, the connection is by radio frequency.

In possible embodiments of the present invention, the at least one first data acquisition device 112 is a data acquisition device of an anesthesia monitoring device 106, such as an anesthesia monitoring device 106 of an anesthesia machine 104. In those modes of implementation, the at least one computing device 114 is connected to an anesthesia monitoring device 106 via which it receives, by means of the sensors 102 connected to it, physiological data of a patient 100 and of their interaction with electromechanical devices to which the patient 100 may be connected.

In preferred embodiments, as illustrated in FIG. 1, the at least one first data acquisition device 112 is independent of a data acquisition device of an anesthesia monitoring device 106.

As also shown in FIG. 1, the data acquisition device 112 is preferably positioned under the surgical table 120. This arrangement and the wireless connection to the computing device 114 avoids having cables that could cause hindrances crossing the site where the anesthesiological procedure is being executed.

In another possible embodiment, wireless sensors 110 are utilized.

In yet another embodiment of the invention, the at least one first data acquisition device 112 is integrated into the at least one computing device 114.

In some embodiments of the present invention, the at least one computing device 114 with at least one first data acquisition device 112 integrated therein can be further integrated into an anesthesia machine 104. Such at least one computing device 114 with at least one first data acquisition device 112 integrated therein may be used as a main monitoring device.

In preferred embodiments, the at least one computing device 114 with at least one first data acquisition device 112 integrated therein is independent of anesthesia monitoring devices 106 and receives data from independent sensors 110.

In other embodiments, the at least one computing device 114 with at least one first data acquisition device 112 integrated therein is independent of anesthesia monitoring devices 106 and receives data from sensors 102 of anesthesia monitors 106 or anesthesia machines 104.

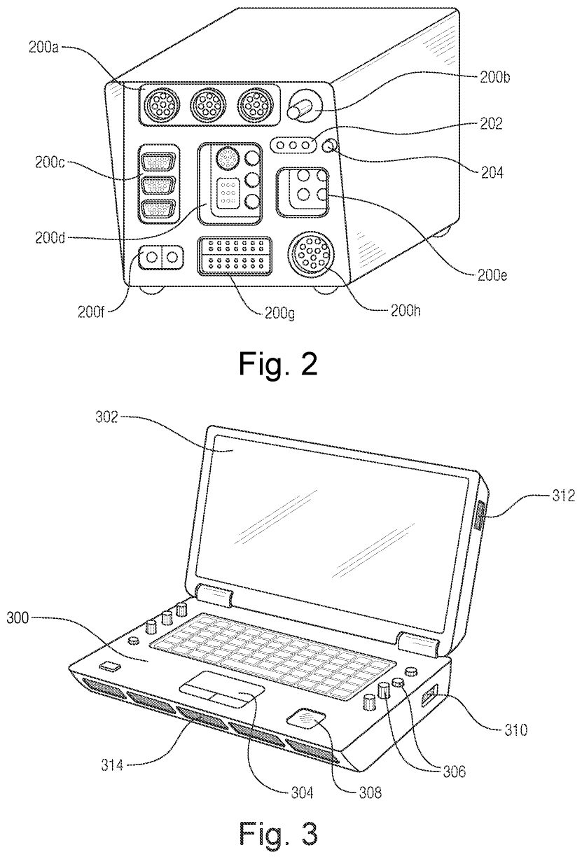

FIG. 2 shows in greater detail a first data acquisition device 112 in accordance with one possible embodiment of the present invention.

As illustrated there, the data acquisition device 112 may comprise channels 200a, 200b, 200c, 200d, 200e, 200f, 200g, 200h to receive the cables from the one or more sensors 110 that measure physiological data of a patient 100 and of their interaction with electromechanical devices to which the patient 100 is connected. In this example, channels 200a are arranged to be connected to invasive pressure measurement systems, channel 200b to a non-invasive blood pressure measurement system, channels 200c to pulse oximeters, channels 200d to airway pressure, flow and volume sensors and gas composition sensors, channels 200e to near-infrared spectrometry data measurement systems, channels 200f to temperature sensors, channels 200g to electroencephalography data measurement systems, and channel 200h to electrocardiography sensors.

Preferably, the data acquisition device 112 is powered by at least one battery so that the patient 100 is galvanically isolated from electrical current, although the use of other internal or external power supplies is contemplated.

In a preferred embodiment, the data acquisition device 112 comprises two interchangeable batteries. This allows recharging one of the batteries while the other one is being utilized so as to have a continuous power supply.

Additionally, the data acquisition device 112 may comprise a temperature sensor capable of detecting an electrical fault.

The at least one first data acquisition device 112 may further comprise activity/charge/low battery indicator lights 202 and a reset button 204.

Examples of the one or more sensors 110 capable of capturing physiological data of a patient 100 and of their interaction with electromechanical devices to which the patient 100 may be connected, compatible with the at least one first data acquisition device 112 of the present invention, comprise, among others, pulse oximeters, electrocardiographs, non-invasive blood pressure measurement systems, invasive pressure measurement systems, electroencephalography data measurement systems, temperature sensors, gas composition sensors (carbon dioxide, oxygen and volatile anesthetics), airway pressure, flow and volume sensors, Near Infrared Spectrometry (NIRS) data measurement systems.

In preferred embodiments of the present invention, the sensors 110 compatible with the system are electronically identifiable and independent of any other monitoring system 106 such as the sensors 102 of anesthesia monitors 106 or anesthesia machines 104. Additionally, the signal processing protocol employed is universal and open source. In this way, it is possible to generate universally comparable readings.

An “electronically identifiable” sensor or device has a passive or active electronic system of model and serial number identification that is recognized by the system, transmitted and stored in at least one main database.

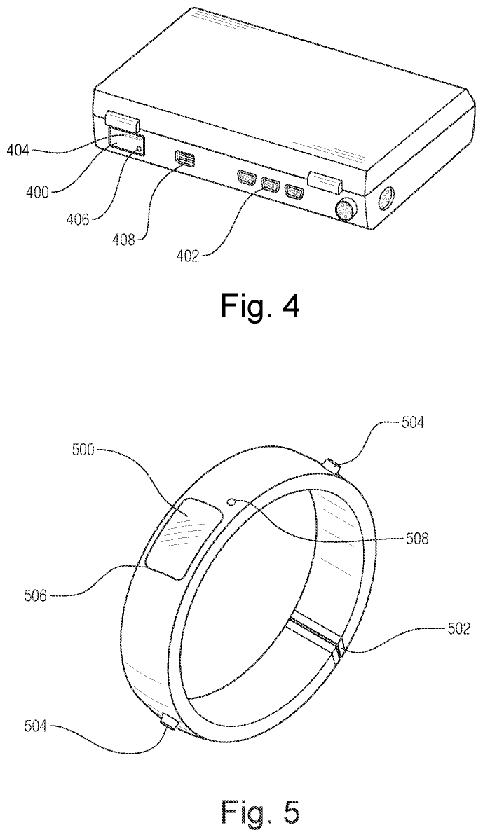

FIG. 3 is a front and right-side perspective view of a computing device 114 according to a possible embodiment of the present invention, where greater details of the same may be observed as it is in its unfolded mode of use.

FIG. 4 is a rear and left-side perspective view of a computing device 114 according to a possible embodiment of the present invention, where greater details of it may be observed as it is folded.

The at least one computing device 114 may be any type of general or specific purpose stationary or mobile computing device, including a mobile computer (e.g., a personal digital assistant (PDA), laptop, notebook computer, tablet computer, netbook, etc), a mobile phone (for instance, a cell phone or smartphone), or a stationary computing device such as a desktop computer or personal computer. In preferred embodiments, the at least one computing device 114 is a general or specific purpose laptop, notebook computer or desktop computer. Exemplary computing device 114 shown in FIGS. 3 and 4 is a special purpose laptop.

Preferably, the at least one computing device 114 is powered by at least one rechargeable battery 400. Even more preferably, it is powered by two rechargeable batteries. In other embodiments, the at least one computing device 114 is connected directly to the supply network from charging port 310 via an electrical cable and plug. The employment of other internal or external power sources is contemplated.

In embodiments, the at least one computing device 114 comprises at least one processor (such as a central processing unit (CPU), a graphics processing unit (GPU)), memory (volatile and/or non-volatile) and bus that couples various components including the memory to at least one processor.

The at least one computing device 114 may also comprise hard disk drives, magnetic storage devices, optical disc drives, solid state drives, connected to the bus by means of interfaces, or other types of hardware-based computer-readable storage media.

A number of program modules may be stored on the hard disk, magnetic disk, optical disk, ROM or RAM. These programs include an operating system, one or more application programs, among others.

A user may enter commands and information into the computing device 114 via input devices such as a keyboard 300 and a pointing device. Other input devices may include a microphone, joystick, antennas, scanner, touch screen 302 and/or touch panel 304, buttons/knobs 306, digital pen, a voice recognition system to receive voice input, a gesture recognition system to receive gesture input, a fingerprint reader 308, or similar devices. These and other input devices may be integrated into the at least one computing device 114 or connected to it in a wired (via one of its communication ports 402) or wirelessly. As an example, they may be connected to the processor by means of a serial port interface that is coupled to the bus, but they may also be connected by other interfaces, such as a parallel port, or a universal serial bus (USB).

Anesthesiologist 118a, 118b may enter supplementary data such as comments, mark events during transmission, enter trademarks and drug batch numbers, etc. via one of the input devices or by sending them by means of at least one other authorized electronic or computing device connected to the main one 114. Supplementary data that may be added prior to the procedure comprise, among others, anthropometric data (such as age, height, weight, race), medical history (such as diseases, allergies, medications, utilization and configuration of implantable devices such as pacemakers). Supplementary data that may arise during the procedure and may be added during it comprises, among others, details of airway management, medication utilized, drug infusions details, blood components administration, detection, evolution and treatments of adverse events, medication details such as batch identification, report of surgical details, laboratory analytics results, intra-surgical diagnostic methods such as ultrasound scans and radioscopy, image recording (photos of test results, laboratory values, anatomical or surgical details).

In preferred embodiments of this invention, the system is compatible with international medical communication standards which are syntactic, such as HL7 (Health Level Seven), so as to facilitate information exchange and/or semantics for the interpretation of the terms utilized in the information exchange. In this way, the at least one computing device 114 is able to receive files (such as text files, image files like photos, audios, or videos) from other medical devices (such as an ultrasound machine) or non-medical devices (such as a mobile phones) authorized and validated by the user.

Additionally, the at least one main computing device 114 may include peripheral output devices such as display screens 302, speakers, and printers connected to the bus via an interface.

The display screen 302 may be external to, or incorporated into, the computing device 114. The display screen 302 may display information, as well as be a user interface for receiving commands and/or other information from the user (e.g., by touch, finger gestures, virtual keyboard, etc.).

In a preferred embodiment of the invention, the display screen 302 renders the data sampled by the first data acquisition device 112 in real time. This makes information generated by the group of independent sensors 110 available to the anesthesiologist 118a, 118b as a backup data set. This may be useful in case of any inconvenience with the anesthesia monitors 106 or anesthesia machines 104 such as: interferences, accidental disconnection of sensors 102 errors of measurement or malfunction of their systems.

The display options may be customizable by anesthesiologist 118a, 118b.

Additionally, the statuses of batteries utilized in the system and the quality of the connections may be rendered on the display screen 302.

In another embodiment, display screen 302 is additionally employed to display a checklist process prior to initiating the anesthesiological procedure. This process comprises steps where check indications are displayed and their confirmation is requested (e.g., check of the anesthesia machine 104, blood availability, patient confirmation). Preferably, so as to move from one step of the process to the next, it is necessary to unlock the “next” button before selecting it, clicking characters that the system randomly highlights with different colours or shapes.

Preferably, the measured variables are depicted on laterally shifted Cartesian axes to show time series or waves and in squares to display intermittent quantitative values. Time series or wave displacement may be paused for analysis and measurements such as width and height of curves, areas under the curve and derivatives, (for example, to measure contractility in invasive blood pressure monitoring or to calculate dead space in volumetric capnography curves).

Additionally, the display screen 302 is capable of rendering non-traditional visualization modes such as “tunnel vision”, three-dimensional beat-by-beat representation of the heart axis, and any user generated data visualization design.

While it is possible to customize the display format to the preference of the anesthesiologist 118a, 118b, it may also be quickly returned to a standard format, for example, by pushing a button. This allows a quick return to a universal standard format in cases where help is required from another colleague for fast interpretation of shown data.

The at least one computing device 114 may include an output peripheral which is a chronometer signal capable of connecting to a display screen. The chronometer may be located or projected in the vicinity of an area of interest that is being recorded by any video camera. As the chronometer signal is transmitted in real time from the computing device 114 to the remote storage system together with the system sampled data, this allows the subsequent synchronization of the recorded video with the sampled data set of the anesthesiological procedure.

Optionally, the at least one computing device 114 comprises at least one input/output port 402 for maintenance or for connection to other devices not belonging to the recording system of the present invention.

In preferred embodiments, the at least one computing device 114 comprises at least one authentication system 308.

To authenticate the identity of the anesthesiologist 118a, 118b who is performing the procedure different subsystems may be employed: subsystems based on something that the anesthesiologist 118a, 118b knows (knowledge factors), such as passwords; subsystems based on something that the anesthesiologist 118a, 118b has (ownership factors), such as smart cards or tokens; subsystems based on something the user is (inherence factors) or subsystems based on biometric authentication. The latter are based on physical features of the user, such as shapes of the face, iris or retina, fingerprints, geometric features of the hands, voice characteristics, or on behavioral features, such as writing and signature. Biometric authentication subsystems are preferably employed, since they are the most secure.

The preferred authentication system 308 is a biometric authentication system (such as fingerprint identification, facial recognition, iris identification, voice recognition). Fingerprint identification using a fingerprint reader 308 incorporated into the computing device 114 is preferred. Authentication may be required at the beginning and end of the procedure. Authentication may also be required sporadically, periodically or in response to events during the procedure.

The at least one computing device 114 has the capability to connect to the Internet via Wi-Fi or mobile network, so as to to receive information and transmit the sampled data in real time to the remote storage system.

Preferably, the at least one computing device 114 makes it possible to determine, by means of the Internet connection, the geolocation and to synchronize the system with Coordinated Universal Time (UTC).

The at least one computing device 114 may additionally comprise one or more of activity/charge/low battery indicator lights 404, charge test button 406, mobile data card socket or slot 408, and air vents 314.

In one embodiment of the present invention, the at least one second data acquisition device 312 is connected to the at least one computing device 114. In another embodiment, as represented in FIG. 3, the at least one second data acquisition device 312 is integrated into the at least one computing device 114. In still other embodiments, the at least one second data acquisition device 312 is connected to, or integrated into, the at least one first data acquisition device 112.

In possible embodiments of the present invention, the at least one second data acquisition device 312 is wirelessly connected, for instance, by radio frequency, to the at least one computing device 114 and/or the at least one first data acquisition device 112.

The at least one second data acquisition device 312 is capable of connecting to one or more sensors that measure environmental conditions in real time at the site where the procedure is performed, such as an operating room. In an embodiment, it is compatible with temperature, pressure and humidity sensors.

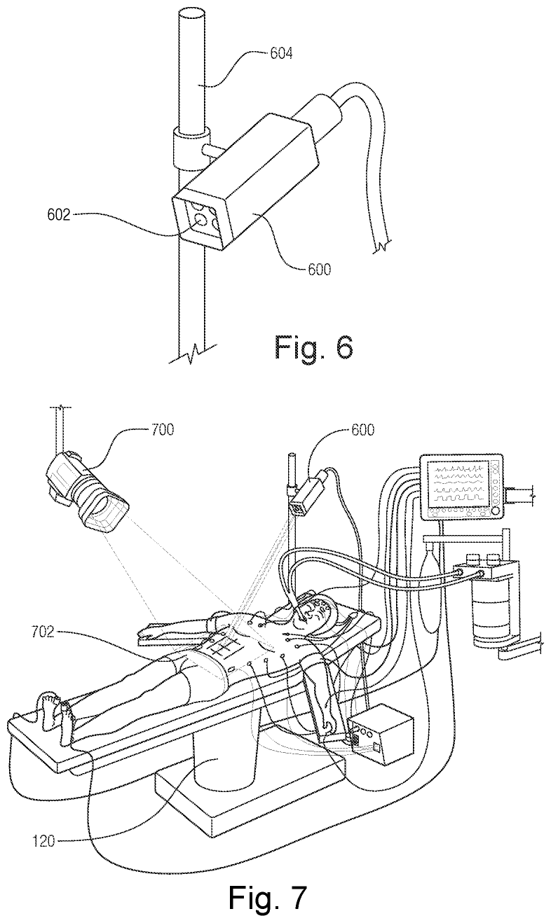

FIG. 5 illustrates in greater detail a portable device 116 in accordance with one possible embodiment of the present invention.

The at least one portable device 116 is an electronic handheld or wearable device that may be carried or worn by the user and, via interaction with the at least one computing device 114 and/or at least one first data acquisition device 112, is configured to generate data related to the temporal-spatial relationship between the at least one anesthesiologist 118a, 118b and the patient 100.

The at least one portable device 116 may be powered by at least one rechargeable battery, although the use of other power sources is contemplated.

In preferred embodiments of the invention, the portable device 116 is a transponder with at least one sensor.

The connection between the at least one electronic device 116 portable by at least one anesthesiologist 118a, 118b and the at least one computing device 114 is preferably wireless, as illustrated in FIG. 1. Still preferably, the communication is by radio frequency (Bluetooth™ or the like).

In embodiments of the present invention, the at least one portable device 116 is connected to the at least one computing device 114 by means of a first wireless connection (preferably, by radio frequency). The data about this connection is sent in real time to the remote storage system.

In some embodiments, the at least one electronic device 116 portable by the at least one anesthesiologist 118a, 118b may also be connected to the at least one first data acquisition device 112. Preferably, the connection is by radio frequency (although other types of connections are contemplated) and the data about the connection is sent in real time to the at least one computing device 114 and, from there, to the remote storage system.

The data about the first wireless connection between the at least one portable device 116 and the at least one computing device 114 and/or the at least one first data acquisition device 112 is useful in determining whether or not the anesthesiologist 118a, 118b is close to the patient 100.

The proximity and/or distance between the at least one portable device 116 and the at least one computing device 114 and/or the at least one first data acquisition device 112 and, therefore, between the anesthesiologist 118a, 118b and their patient 100 is estimated based on the signal intensity and/or quality of the first wireless connection between the devices.

The proximity estimation consists of determining if an anesthesiologist 118a, 118b is near or far from the patient 100 based on whether the portable device 116 linked to the anesthesiologist 118a, 118b is connected via a first wireless connection to at least one computing device 114 and/or to the at least one first data acquisition device 112. The coverage of the first wireless connection has a range coinciding with a maximum distance that ensures that the anesthesiologist 118a, 118b is able to properly monitor the patient 100 and their surroundings. Having exceeded that coverage radius, the signal begins to weaken until it is lost.

In preferred embodiments, the first wireless connection between the at least one portable device 116 and the at least one computing device 114 and/or the at least one first data acquisition device 112 has a coverage radius of approximately 6 meters.

The distance estimation consists of a quantitative estimation based on some indicator of the intensity and/or quality of the signal of the first wireless connection between the devices. Examples of quantitative estimation methods of the distance between devices that may be applied in the present invention are based on the RSS (Received Signal Strength) parameter, which is an indicator of the received signal strength at the device's antenna; based on the ToA (Time of Arrival) which is an indicator of the arrival time of a signal; based on the TDoA (Time Difference of Arrival) which is an indicator of the difference in arrival times of two signals, each with different propagation speeds; and based on the AoA (Angle of Arrival) utilized in addition to other parameters, which is an indicator of the angle of arrival that forms the direction of propagation of an incident wave and a certain reference direction.

In embodiments of the present invention, data about the first wireless connection between the at least one portable device 116 and the at least one computing device 114 and/or the at least one first data acquisition device 112, in particular, data on the intensity and/or quality of the signal of that first wireless connection, is transmitted in real time to the remote storage system, thus continuously sampling and recording the proximity and/or distance between the at least one portable device 116 and the at least one computing device 114 and/or the at least one first data acquisition device 112 and, therefore, between the anesthesiologist 118a, 118b and their patient 100.

In preferred embodiments of the invention, the at least one electronic device 116 portable by the at least one anesthesiologist 118a, 118b comprises at least one motion sensor capable of detecting presence or absence of movement signals from that portable device 116. In even more preferred embodiments, the motion sensor is additionally capable of differentiating between regular and irregular movements within a given period of time.

In possible embodiments of the invention, the system is capable of identifying and classifying movements from the at least one portable device 116 as regular or irregular. Preferably, this identification and classification is carried out on the at least one portable device 116 or on the at least one computing device 114. When identifying an irregular movement, the system may interpret that it comes from the anesthesiologist 118a, 118b who is employing the portable device 116 as human movements are mostly irregular. In case that continuous regular movement is detected, the system may interpret that the portable device 116 is not being carried by an anesthesiologist 118a, 118b and may be supported over a machine or electromechanical device which produces repetitive movements or vibrations. Examples of motion sensors are accelerometers, gyroscopes, magnetometers, vector rotation sensors, gravity sensors, and linear acceleration sensors. Preferably, the at least one portable device 116 comprises at least one accelerometer.

The data measured by the at least one motion sensor is transmitted in real time by the first wireless connection between the at least one portable device 116 and the at least one computing device 114.

In embodiments of the present invention, the at least one portable device 116 is also connected to the at least one computing device 114 by means of a second wireless connection, which is a short-range connection, (preferably by radio frequency) when the at least one portable device 116 is at a distance from the at least one computing device 114 equal to or less than a value considered suitable for user manipulation. Information about the identity of the at least one portable device 116 that is in close proximity is transmitted by this second connection to the at least one computing device 114.

A distance suitable for user manipulation is a short distance such as to ensure that the anesthesiologist 118a, 118b carrying the portable device 116 is in close contact with the computing device 114, that is, close enough to the computing device 114 to be able to manipulate it. An example of a distance suitable for user manipulation is a distance equal to or less than approximately 100 cm.

In such embodiments, the at least one computing device 114 is capable of sending data on its manipulation together with an indication of whether at the time of the manipulation at least one portable device 116 was connected to it 114 via the second short-range wireless connection and, if so, sending by this method the identity of that at least one portable device 116 associated with a single anesthesiologist 118a, 118b. “Manipulation of the computing device 114” should be understood as any interaction of a user with that device 114 by means of an input device thereof 114.

In some embodiments, the at least one computing device 114 is additionally configured to unlock some of its functionalities when at least one portable device 116 is identified at a distance equal to or less than a value considered suitable for user manipulation. This ensures that only the anesthesiologist 118a, 118b who is performing the procedure is the one who may unlock those lockable functionalities of the computing device 114. Examples of lockable functionalities are manual input of data, customization of displayed data on the at least one display screen 302 of the computing device 114, measurements and navigation/exploration of curves and graphs generated with sampled data 110, muting or decreasing the volume of the computing device 114 and conducting a query on a patient medical history 100.

In other embodiments, the at least one computing device 114 is further configured to unlock some of its functionality via an authentication action on the same computing device 114. The way an anesthesiologist 118a, 118b unlocks a lockable functionality is also transmitted and registered in the at least one main database.

In preferred embodiments, the second wireless connection is a short-range radio frequency connection and the at least one portable device 116 is also an RFID (radio frequency identifier) transponder. This transponder may be passive, active or a combination thereof. Each electronic device 116 portable by an anesthesiologist 118a, 118b in the system is associated with a single anesthesiologist 118a, 118b. Furthermore, the at least one computing device 114 additionally comprises a short-range RFID transmitter/receiver capable of capturing the RFID transponder signal and extracting and transmitting the information contained therein when the distance is equal to or less than a value considered suitable for user manipulation.

In even more preferred embodiments, the at least one portable device 116 is a transponder with sensors.

In embodiments of the invention, the at least one portable device 116 may comprise at least one microcontroller capable of providing additional functionalities.

In preferred embodiments, the at least one electronic device 116 portable by the at least one anesthesiologist 118a, 118b may comprise at least one authentication system 500.

The preferred authentication system 500 is a biometric authentication system (such as fingerprint identification, facial recognition, iris identification, voice recognition). Fingerprint identification using a fingerprint reader 500 incorporated into the portable device 116 is preferred. Authentication may be required at the beginning and end of the procedure. Authentication may also be required sporadically, periodically or in response to events during the procedure.

In some embodiments, such events may be:

detection of weak connection or loss of connection between the portable device 116 and the computing device 114 and/or the first data acquisition device 112;

detection of absence of portable device 116 movement;

detection of regular movements of the portable device 116; and

detection that the portable device 116 has been removed from the anesthesiologist's body 118a, 118b.

It should be understood that the fact that an anesthesiologist 118a, 118b does not authenticate after the system request does not imply a negative connotation per se, but only generates statistical data. As an example, a record in which authentication was requested on the portable device 116 4 times and 3 of them were effective, may reflect that the anesthesiologist 118a, 118b was busy performing, for example, a sterile procedure without wearing the device 116 at the time the authentication failed. Conversely, a 6-hour record in which authentication was requested 6 times and was never performed, added to the fact that the system did not measure activity of the portable device 116 via motion sensors or did not detect its proximity evaluating the quality of connection, could indicate a negative connotation such as, for example, poor patient surveillance performance by the anesthesiologist 118a, 118b. However, it should be understood that for a correct interpretation of any of these events it is necessary to evaluate and integrate all the measurements of the record.

In preferred embodiments, the data related to the temporal-spatial relationship between the at least one anesthesiologist 118a, 118b and the patient 100 and, therefore, to the quality of surveillance provided by the at least one anesthesiologist 118a, 118b to the patient 100, generated by the interaction between the at least one portable device 116 and the at least one computing device 114 and/or the at least one first data acquisition device 112 comprises one or more of:

data on the proximity and/or distance between the at least one portable device 116 and the at least one computing device 114 and/or the at least one first data acquisition device 112 and, therefore, between the anesthesiologist 118a, 118b and their patient 100 based on the intensity and/or quality of the signal of the first wireless connection between the devices;

data on the activity and/or inactivity of the anesthesiologist 118a, 118b measured by means of the at least one motion sensor of the at least one portable device 116, detecting the presence or absence of movement;

authentication data of the anesthesiologist 118a, 118b in the at least one computing device 114 and/or in the at least one portable device 116; and

data on authenticated manipulations of the at least one computing device 114 and/or of the at least one portable device 116.

In preferred embodiments, the at least one portable device 116 communicates to the at least one computing device 114 and/or to the at least one first data acquisition device 112 via a first wireless connection, preferably by radio frequency, such as Bluetooth, and, based on the intensity and/or quality of the signal of that connection, data is generated on the proximity and/or distance between the at least one identified portable device 116 and the at least one computing device 114 and/or the at least one first data acquisition device 112 and, therefore, between the anesthesiologist 118a, 118b and their patient 100. Furthermore, the data on the activity and/or inactivity of the anesthesiologist 118a, 118b measured via the at least one motion sensor of the at least one portable device 116 and the authentication data in that portable device 116 is transmitted to the at least one computing device 114 via the first wireless connection. Furthermore, data on the identity of the at least one portable device 116 in close proximity to the at least one computing device 114 is transmitted to it by a second wireless connection, which is a short-range connection, preferably by radio frequency. This data on the identity of the at least one portable device 116 in close proximity to the at least one computing device 114, considered in conjunction with the manipulation data of the at least one computing device 114, makes it possible to associate a user 118a, 118b of a portable device 116 as the executor of a manipulation in the at least one computing device 114.

In these embodiments, the data related to the temporal-spatial relationship between the at least one anesthesiologist 118a, 118b and the patient 100 and, therefore, to the quality of surveillance provided by the at least one anesthesiologist 118a, 118b to the patient 100, generated via the interaction between the at least one portable device 116 and the at least one computing device 114 and/or the at least one first data acquisition device 112 also comprises data on the identity of at least one portable device 116 in close proximity to the at least one computing device 114 in such a way as to be able to associate a manipulation of the at least one computing device 114 with an anesthesiologist 118a, 118b if at the time of that manipulation at least one portable device 116 was connected to it via the second wireless connection.

When the second wireless connection is by short-range radio frequency, the identity of the at least one portable device 116 associated with a single anesthesiologist 118a, 118b is detected through the interaction between the RFID transponder of the at least one portable device 116 and the RFID transmitter/receiver of the at least one computing device 114.

In preferred embodiments of the present invention, the electronic device 116 portable by at least one anesthesiologist 118a, 118b comprises:

at least one first wireless communication system capable of connecting to at least one computing device 114 and/or at least one first data acquisition device 112 by means of a first wireless connection and of transmitting through it data on the identity of the portable device 116 and data on the proximity and/or distance between the portable device 116 and the at least one computing device 114 and/or the at least one first data acquisition device 112 and, therefore, between the anesthesiologist 118a, 118b and their patient 100 based on the intensity and/or quality of the signal of the first wireless connection between the devices;

at least one motion sensor capable of detecting movement signals from the anesthesiologist 118a, 118b; and

at least one authentication system 500 capable of verifying the identity of a user 118a, 118b carrying the portable device 116 in response to receiving an authentication request from the at least one computing device 114,

wherein the data on the activity and/or inactivity of the anesthesiologist 118a, 118b measured by the at least one motion sensor and the authentication data is transmitted to the at least one computing device 114 via the first wireless connection.

In even more preferred embodiments, the electronic device 116 portable by at least one anesthesiologist 118a, 118b further comprises at least one second wireless communication system capable of connecting to at least one computing device 114 by means of a second short-range wireless connection and of transmitting data on the identity of that portable device 116.

In some embodiments, the electronic device 116 portable by at least one anesthesiologist 118a, 118b further comprises a vibration system and/or an audible alarm system.

The at least one computing device 114 may send to the at least one portable device 116 one or more of the following communications: instruction to emit vibration and/or audible alert and/or alarm, request for authentication, first wireless connection signal quality and strength and second wireless connection signal quality and strength. In turn, the at least one portable device 116 may send to the at least one computing device 114 one or more of the following communications: authentication data, movement data, manipulations on the portable device 116, portable device identity data, first wireless connection signal quality and strength and second wireless connection signal quality and strength.

Therefore, the system of the present invention is configured to generate and record data related to the temporal-spatial relationship between the at least one anesthesiologist 118a, 118b and the patient 100 which may allow the estimation of the quality of surveillance provided by the anesthesiologist 118a, 118b by considering the proximity and/or distance between the portable device 116 (and, therefore, the anesthesiologist 118a, 118b) and the computing device 114 and/or the first data acquisition device 112 (and, therefore, the patient 100). Furthermore, the system of the present invention is capable of measuring and recording the latency between the detection of an event and the implementation of an action and also between the issuance of warnings and/or alarms and their attendance or dismissal via actions taken on the computing device 114 and/or portable device 116.

By measuring the proximity and/or distance between the portable device 116 by the anesthesiologist 118a, 118b and the computing device 114 and/or the first data acquisition device 112, it may be estimated whether the anesthesiologist 118a, 118b maintains an active surveillance of the patient 100. As previously mentioned, “active surveillance” refers to being attentive to the patient 100 and the anesthesia monitors 106 by staying close to them and in direct visual contact. In contrast, a “passive surveillance” refers to only paying attention to configured alarms.

Additionally, by measuring the latency in the action of the anesthesiologist 118a, 118b, it is possible to obtain an estimate of their surveillance efficiency.

In further embodiments, the quality of surveillance of the anesthesiologist 118a, 118b to the patient 100 may be also estimated by considering the quality of scientific data generated by the anesthesiologist 118a, 118b based on the quantity and quality of supplementary data submitted by them into the system through the computing device 114.

In preferred embodiments, the at least one electronic device 116 portable by the at least one anesthesiologist 118a, 118b, which, by interacting with the at least one computing device 114 and/or the at least one first data acquisition device 112, is configured to generate data related to the temporal-spatial relationship between the at least one anesthesiologist 118a, 118b and the patient 100 and, therefore, to the quality of surveillance provided by the at least one anesthesiologist 118a, 118b to the patient 100, is a wearable device.

Wearable device refers to any wearable technological device, that is, a technological device that is incorporated into clothing or accessories capable of continuously interacting with the user and with at least one other device so as to perform some specific function. Preferably, the wearable device is an accessory such as an electronic bracelet, watch, or ring.

In a preferred embodiment of the present invention, as illustrated in FIGS. 1 and 5, the wearable device 116 is an electronic bracelet. Preferably, the electronic bracelet 116 is made of materials (such as silicone, rubber or fabric) and has a configuration (for example, airtight) that makes it easily sanitizable.

In exemplary embodiments of the present invention where the portable device 116 is a wearable device, the system detects when it is removed from the wearer's body and when it is put back on. In embodiments, detection is performed by the sudden acceleration pattern that is generated when the wearable device 116 is removed from the body.

In still other embodiments, the wearable device 116 is attached to the body of the anesthesiologist 118a, 118b by an opening and closing fastener provided with an electronic contact 502. In this way, the system may interpret that the wearable device 116 has been removed from the anesthesiologist's 118a, 118b body when the circuit is opened and that it has been repositioned when the circuit is closed.

Preferably, the system detects when the device 116 is removed from the body or attached to it by the opening of the electronic circuit and/or by the sudden acceleration pattern that is generated when opening and closing the device 116.

In embodiments of the invention, the system requests authentication from the anesthesiologist 118a, 118b on this wearable device 116 in response to detecting that the device 116 has been removed from their body.

The portable device 116 may further comprise one or more of the following: a plurality of buttons 504; an interface 506; a vibration system; an audible alarm system; a microphone; activity/charge/low battery indicator lights 508.

In addition to the functionality of measuring variables on the quality of surveillance performed by the anesthesiologist 118a, 118b on the patient 100, the portable device 116 may function as a safety system in at least the following cases:
In response to the portable device 116 detecting the absence of movement or detecting regular movements in it 116 and/or weak connection or absence of a first wireless connection with the computing device 114 and/or the first data acquisition device 112, for a period of time greater than a pre-established time considered prudential, a vibratory signal is emitted. A “time considered prudential” should be interpreted as a period of time during which the patient 100 may remain without active surveillance without compromising their safety. By way of example, a prudential period of time is in the range of about 1 to about 3 minutes, in particular, in the range of about 1 to about 2 minutes.
In response to the portable device 116 detecting the absence of movement or detecting regular movements in it 116 and/or weak connection or absence of a first wireless connection with the computing device 114 and/or the first data acquisition device 112, for a period of time greater than a pre-established time considered unsafe for the patient, an audible signal is emitted. A time considered “unsafe for the patient” should be interpreted as a period of time after which the safety of the patient 100 would be seriously compromised without active surveillance. By way of example, a unsafe period of time is in the range of about 3 to about 6 minutes, in particular, in the range of about 4 to about 5 minutes.
In response to the computing device 114 detecting that an inactivity or imprudent distance condition of the portable device 116 persists, it may emit an audible signal as a warning to third parties.

In this way, events such as fainting or inadvertent sleep of the anesthesiologist 118a, 118b in charge of the patient may be warned 100.

Furthermore, the plurality of buttons 504 of the portable device 116 may be programmed for remote control of the computing device 114 in terms of certain actions such as dismissing warnings and/or silencing alarms. This provides the convenience that the anesthesiologist 118a, 118b does not have to approach the computing device 114 to execute these actions. Preferably, it is necessary to activate at least two buttons 504 simultaneously to send an instruction, so as to avoid inadvertent actuation. In an embodiment, as illustrated in FIG. 5, the number of buttons 504 is two.

In one embodiment, the vibratory system may be further configured to emit vibratory pulses in sync with the patient's sampled signals such as pulse rate, or to emit vibratory pulses of different frequencies or patterns in response to the detection of irregular patient signals (such as arrhythmias) or patient variables that may be out of the normal range (such as blood pressure, temperature, etc).

In embodiments of the present invention, the at least one electronic device 116 portable by the at least one anesthesiologist 118a, 118b additionally comprises an interface for the anesthesiologist 118a, 118b to interact with the at least one computing device 114. The referred interface may be a scrollable interface 506.

In some possible embodiments of the present invention, the at least one portable device 116 is capable of capturing biometric data from the at least one anesthesiologist 118a, 118b who carries it, such as pulse oxymetry, electrocardiography, electroencephalography, among others.

It should be noted that, in a preferred embodiment of the invention, all the aforementioned actions and communications between the at least one portable device 116 and the at least one computing device 114 and/or the at least one first data acquisition device 112 are transmitted in real time and they are recorded in the main database.

In one embodiment of the invention, the system comprises a plurality of portable devices 116, one utilized by a main anesthesiologist 118a and at least one other utilized by a collaborating anesthesiologist or anesthesiologist in training 118b, being the portable device 116 of the main anesthesiologist 118a in communication with each of the portable devices 116 of collaborating anesthesiologists or anesthesiologists in training 118b and, in turn, each portable device 116 being also in communication with the at least one computing device 114. When there are only two portable devices 116, they establish a communication with each other and with the computing device 114 using a peer to peer network topology as shown in FIG. 1. The distance between the portable devices 116 is estimated by measuring the signal quality and strength between the portable devices and this data is also transmitted in real time to the computing device 114.

Depending on the experience of the anesthesiologists in training 118b, the system may be configured to consider as normal the sole presence in the operating room of only them 118b during certain periods, that is, without the supervision of the main anesthesiologist 118a. Otherwise, the system may be configured to emit a first warning sign if the main anesthesiologist 118a is not in the operating room for a period of time considered detrimental for patient safety. Additionally, the system may be configured to emit at least a second warning signal when the main anesthesiologist 118a is not in the operating room for a period of time considered unsafe. In embodiments, that at least one second warning signal is different from the first one. The employment of as many portable devices 116 as main anesthesiologists 118a and collaborators or in the process of training 118b are involved in the procedure is contemplated.

In situations of long procedures where a replacement of anesthesia professionals is desired (for example, in transplant procedures, commando surgeries), the substitution may be implemented via cross-authentication in the computing device 114 and portable devices 116 of each anesthesiologist 118a, 118b.

Preferably, each computing device 114, portable device 116 and data acquisition devices 112, 312 of the registration system of the present invention has a passive or active electronic system for identifying the model and serial number that is transmitted to the at least one database.

In embodiments of this invention, the system is compatible with “Closed-Loop” anesthesia infusion systems, that is, automatic drug infusion systems that adapt the infusions rates by reading physiological data feed. The sampled and transmitted data by the system of the present invention has characteristics (universality, traceability, authenticity, autonomy, real-time transmission, decentralized storage) which allow it to be employed not only as input/data feed for such Closed-Loop control systems, but as an immutable record of the effectiveness and efficiency of such feedback control systems. In this manner, the performance of such systems is transparently safeguarded, allowing audit and preventing the manipulation by any third parties.

The sampled and transmitted data by the system of the present invention, by virtue of its aforementioned characteristics, may be ideal as real time data feed for control systems based on Artificial Intelligence.

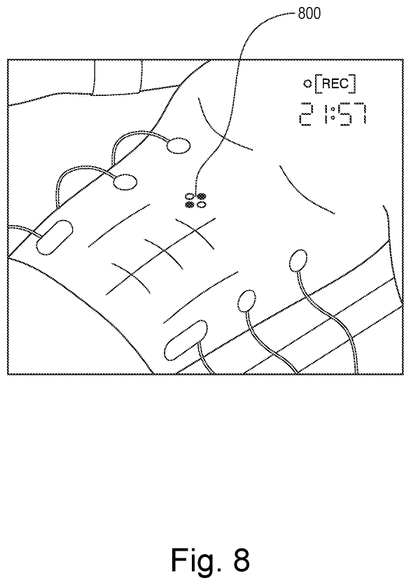

In another embodiment illustrated in FIGS. 6 to 8, the recording system comprises at least one video synchronization device 600 connected to the at least one computing device 114.

FIG. 6 shows a perspective view of the video synchronization device 600 in detail, where that device 600 is mounted by way of example on a post 604, preferably a saline stand, which is installed next to the surgical table 120.

FIG. 7 illustrates the implementation of the video synchronization device 600 in the recording system of the present invention when using a video camera 700 to record an area of interest 702.

FIG. 8 shows in detail the area of interest 702 that is being recorded by the video camera 700.

The video synchronization device 600 comprises an array of a plurality of lasers 602 (preferably four or more) that are projected alternately in the vicinity of an area of interest 702 that is being recorded by any video camera 700, generating a dynamic and variable light code 800. That projected code 800 is also electronically transmitted in real time to the computing device 114 and, from there, to the remote storage system together with the general dataset, allowing the subsequent synchronization of the recorded video with the recording of the anesthesiological data of the procedure.

Preferably, the connection between the video synchronization device 600 and the at least one computing device 114 is wired. In embodiments, the synchronization device 600 is powered by the at least one computing device 114 to which it is connected.

In some embodiments, the video synchronization device 600 additionally comprises lights to indicate its correct connection, operation and malfunction.

In preferred embodiments, the video synchronization device 600 additionally comprises a passive or active electronic system with which its model and serial number may be identified when connected to the at least one computing device 114.

In embodiments of the present invention, the system is capable of recording audio data in the at least one main database by means of a microphone incorporated into the computing device 114, a microphone incorporated into the portable device 116, a microphone incorporated into the first 112 or second 312 data acquisition device, an external microphone connected to the computing device 114 or a microphone incorporated into an authorized external device connected to the computing device 114. Examples of audio data that may be recorded are voice recordings of patients 100 (or their relatives in charge) giving consent for the recording of data, audios of anesthesiologists 118a, 118b as part of the supplementary data provided during anesthesiological procedures, among others.

It should be understood that the connection between devices of the present invention must be construed broadly and capable of including, among others, pairing and synchronization, if applicable.

In a second aspect, the present invention is directed to a method for recording anesthesiological procedures which are performed in an operating room comprising the steps of:

receiving in the at least one computing device 114 a login credential from at least one anesthesiologist 118a, 118b who accesses the at least one computing device 114 and, in response to the authentication of the anesthesiologist 118a, 118b via an authentication system 308:

generating at least one file to contain data on the anesthesiological procedure;

receiving in the at least one computing device 114 preliminary data about the patient 100 and/or about the procedure to be performed, via an input device connected to, or integrated into, it, and send that preliminary data to the remote storage system;

receiving confirmation from the at least one anesthesiologist 118a, 118b, via authentication on the at least one computing device 114 and/or the at least one portable device 116, that the anesthesiological procedure is about to begin;

in response to receiving confirmation that the anesthesiological procedure is about to begin, start sampling physiological data by means of sensors 110 and data related to the temporal-spatial relationship between the at least one anesthesiologist 118a, 118b and the patient 100 and, therefore, to the quality of surveillance provided by the at least one anesthesiologist 118a, 118b to the patient 100, via the interaction between the at least one portable device 116 with the at least one computing device 114 and/or the at least one first data acquisition device 112, and transmitting in real time such sampled data from the at least one first data acquisition device 112 and portable device 116 to the at least one computing device 114 and from there to the remote storage where an immutable copy of the sampled and preliminary data that are linked to the at least one generated file is stored;

receiving confirmation from the at least one anesthesiologist 118a, 118b, by authentication on the at least one computing device 114 and/or the at least one portable device 116, that the anesthesiological procedure has ended; and

in response to receiving confirmation of completion of the anesthesiological procedure, finishing the transmission and recording of data.

It should be understood that some of the steps in this method could occur in another order, or even simultaneously, without departing from the spirit and scope of the present invention.

In embodiments, the step of generating a file for containing data about the anesthesiological procedure is performed in the remote storage system, via the at least one computing device 114. In other embodiments, that step of generating a file for containing data about the anesthesiological procedure is executed in the at least one computing device 114 and, in such cases, the method further comprises the subsequent step of sending the generated file to the remote storage system.

In possible embodiments of the present invention, after the generation of a file for containing data about the anesthesiological procedure, the method may further comprise the steps of:

performing a query about the existence of an anesthesiological history of the patient 100, via a query execution module of the system;

in response to receiving a confirmation of the existence of an anesthesiological history of the patient 100, linking the generated file to that history;

in response to receiving a confirmation of the non-existence of an anesthesiological history of the patient 100, creating a history for the patient 100 and linking the generated file to the history.

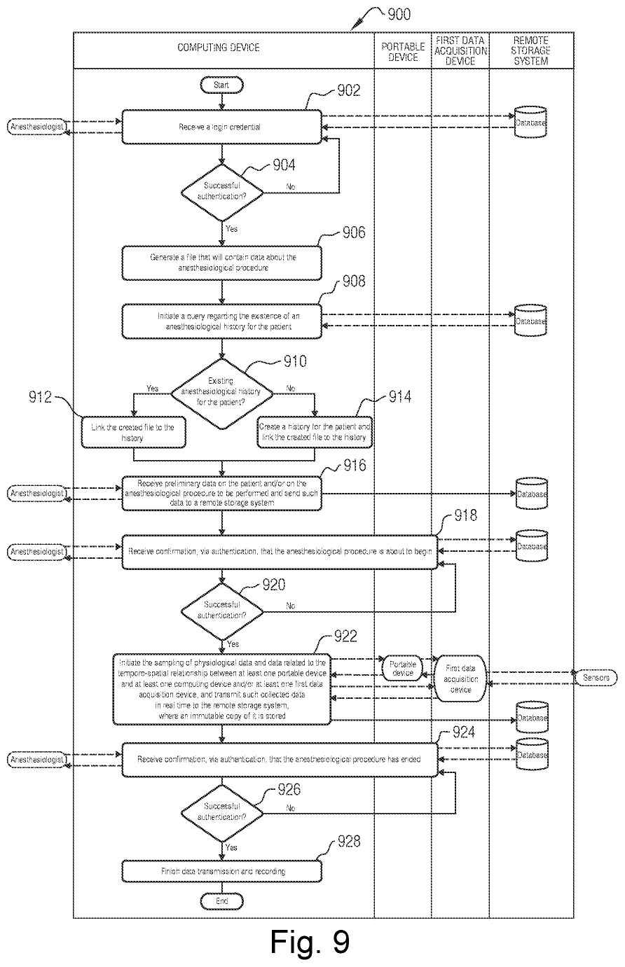

FIG. 9 shows a flowchart depicting a method 900 for recording anesthesiological procedures in accordance with one embodiment of the present invention.

Method 900 begins with step 902. In step 902, a login credential from an anesthesiologist 118a, 118b is received on the computing device 114. In step 904, if the received credential is verified, it is determined that the authentication of the anesthesiologist 118a, 118b has been successful. Credentials may be verified by any of the methods known in the art. If the authentication was successful, the operation proceeds to step 906. Otherwise, it returns to step 902.

In step 906, a file is generated that will contain data about the anesthesiological procedure.

From step 906 the method proceeds to the sequence of steps 908 to 914 which is optional, otherwise, directly to step 916.

In step 908, a query is initiated based on the data stored in the secondary database regarding the existence of an anesthesiological history for patient 100.

In step 910, based on the data returned by the query, it is determined whether there is an existing anesthesiological history for the patient 100. If it exists, the operation proceeds to step 912. Otherwise, the operation proceeds to step 914.

In step 912, the file created in step 906 is linked to the history returned in the query in step 908.

In step 914, a history is created for patient 100 and the file created in step 906 is linked to the history.

In step 916, preliminary data on the patient and/or on the anesthesiological procedure to be performed are received from the anesthesiologist 118a, 118b and that data is sent to the remote storage system.

In step 918, confirmation is received from the anesthesiologist 118a, 118b, via their authentication on the computing device 114 and/or the portable device 116, that the anesthesiological procedure is about to begin. The received authentication data is compared with the data previously stored in the secondary database.

In step 920, if the authentication data received in step 918 is verified by any of the methods known in the art, it is determined that the authentication of the anesthesiologist 118a, 118b has been successful. If the authentication was successful, the operation proceeds to step 922. Otherwise, it returns to step 918.

In step 922, the sampling of physiological data and data from the electromechanical devices connected to the patient via sensors 110 and data related to the temporo-spatial relationship between the at least one anesthesiologist 118a, 118b and the patient 100 and, therefore, to the quality of surveillance provided by the at least one anesthesiologist 118a, 118b to the patient 100, via the interaction between the at least one portable device 116 with the at least one computing device 114 and/or the at least one first data acquisition device 112, is started, and that collected data is transmitted in real time from the at least one first data acquisition device 112 and portable device 116 to the at least one computing device 114 and from this to a remote storage system, where an immutable copy of it is stored.

In step 924, confirmation is received from the anesthesiologist 118a, 118b, via their authentication on the at least one computing device 114 and/or the portable device 116, that the anesthesiological procedure has ended. The received authentication data is verified by any of the methods known in the art.

In step 926, if the authentication data received in step 924 is verified, it is determined that the authentication of the anesthesiologist 118a, 118b has been successful. If the authentication was successful, the operation proceeds to step 928. Otherwise, it returns to step 924. Preferably, after a predetermined number of failed authentication attempts, the method proceeds to step 928, but leaving an indication in the record that it was completed without authentication.

In step 928, the data transmission and recording is completed.

Preferably, the data received about the patient 100 before starting the anesthesiological procedure is the patient's anthropometric data (such as age, height, weight, race) and other relevant characteristics (medical history details, current medications, previous surgeries, allergies, etc.). However, to ensure privacy, no type of personal identification data (such as name, surname, identification numbers) is loaded.

The method may further comprise the step of performing a checklist sub-process consisting of displaying a plurality of check indications on the computing device 114 and requesting confirmation of compliance by the anesthesiologist 118a, 118b.

The method may further comprise the step of geolocation and synchronization with the Coordinated Universal Time (UTC) and requesting the anesthesiologist 118a, 118b to confirm such data, after logging in.

The method of the present invention may further comprise the step of receiving supplementary data input during the anesthesiological procedure.

The method may further comprise the step of stochastically requesting the authentication of the at least one anesthesiologist 118a, 118b in one or more of: at least one portable device 116 and at least one computing device 114, via an authentication system 500, 308, respectively.

The method may further comprise the step of requesting the authentication of the at least one anesthesiologist 118a, 118b in one or more of: at least one portable device 116 and at least one computing device 114, via an authentication system 500, 308, respectively, in response to specific events.

The method may further comprise the step of increasing the frequency of the stochastic request for authentication from the at least one anesthesiologist 118a, 118b in one or more of: at least one portable device 116 and at least one computing device 114, via an authentication system 500, 308, respectively, in events where the system may require further evidence of user authentication or active presence verification.

When the at least one portable device 116 is a wearable device that is attachable to the body of the anesthesiologist 118a, 118b via an opening and closing fastener provided with an electronic contact 502, the method may additionally comprise the step of requesting, by means of an authentication system 500, the authentication of the anesthesiologist 118a, 118b on the wearable device 116 in response to detecting the opening and/or closing of that fastener. Furthermore, it may comprise the step of increasing the frequency of stochastic authentication requests for the anesthesiologist 118a, 118b on the wearable device 116 in response to repeatedly detecting the opening and/or closing of the fastener.

When the at least one portable device is a wearable device 116 that comprises at least one motion sensor capable of detecting movement signals from the anesthesiologist 118a, 118b, the method may further comprise at least one of the following steps:

requesting, via an authentication system 500, the anesthesiologist's authentication on the wearable device 116 in response to detecting a sudden movement that could indicate that the wearable device 116 was removed from the anesthesiologist's body;

increasing the frequency of stochastic authentication requests from the anesthesiologist 118a, 118b on the wearable device 116 in response to repeatedly detecting a sudden movement that could indicate that the wearable device 116 was removed from the anesthesiologist's body;

requesting, via an authentication system 500, the authentication of the anesthesiologist 118a, 118b on the wearable device 116 in response to detecting the absence of movement or detecting regular movements thereof for a period of time greater than a pre-established time considered prudential; and

increasing the frequency of stochastic authentication requests for the anesthesiologist 118a, 118b on the wearable device 116 in response to repeatedly detecting the absence of movement or detecting regular movements thereof for a period of time greater than a pre-established time considered prudential.

When the connection of the at least one portable device 116 with the at least one first data acquisition device 112 and/or the at least one computing device 114 is a first wireless connection, the method 900 may further comprise the step of requesting, by a system of authentication 500, 308, respectively, the authentication of the at least one anesthesiologist 118a, 118b in one or more of: at least one portable device 116 and at least one computing device 114, in response to detecting a weak connection or loss of connection between the at least one portable device 116 and the at least one computing device 114 and/or the at least one first data acquisition device 112 for a period of time greater than a pre-established time considered prudential. Additionally, the method 900 may further comprise the step of increasing the frequency of stochastic authentication requests for the at least one anesthesiologist 118a, 118b in one or more of: at least one portable device 116 and at least one computing device 114, in response to repeatedly detecting a weak connection or loss of connection between the at least one portable device 116 and the at least one computing device 114 and/or the at least one first data acquisition device 112 for a period of time greater than a pre-established time considered prudential.

When the at least one portable device 116 additionally comprises a vibration system and at least one motion sensor capable of detecting movement signals from the anesthesiologist 118a, 118b and the connection of the at least one portable device with the at least one first data acquisition device 112 and/or the at least one computing device 114 is a first wireless connection, the method 900 further comprises the step of emitting a vibratory signal via the at least one portable device 116 in response to it detecting the absence of movement or detecting regular movements in it 116 and/or a weak connection or loss of connection with the at least one computing device 114 and/or the at least one first data acquisition device 112, for a period of time greater than a pre-established period considered prudential. Furthermore, the method may comprise the step of emitting an audible signal via the alarm system of the at least one portable device 116 in response to it detecting the absence of movement or detecting regular movements in it 116 and/or a weak connection or loss of connection between the at least one portable device 116 and the at least one computing device 114 and/or the at least one first data acquisition device 112, for a period of time greater than a pre-established period considered unsafe.

In any of the above cases, the increase in the frequency of the stochastic authentication requests may be progressive as long as the condition persists.

In a preferred embodiment of the present invention, the sampled data that allows keeping a record for reconstructing an anesthesiological procedure is stored in an immutable way in at least one architecturally decentralized database belonging to a remote storage system. An architecturally decentralized storage implies the storage of data in a plurality of storage media in different geographical locations that communicate via a computer network. Preferably, this at least one database is politically and architecturally independent from any third party such as medical institutions, companies that develop monitoring technology or medical equipment. In this way, data is protected from any potential subject or entity that may have intentions to modify any sampled information for their own benefit.

The remote storage system of the present invention comprises at least one remote storage medium, that is, physically located outside the place (offsite) where the data is sampled (hospital). The data sampled during an anesthesiological procedure is transmitted and stored in real time on at least one storage medium that is geographically separated from the place where the data originated.

The at least one remote storage medium may host at least one database.

In embodiments of the present invention, the remote storage system comprises two subsystems with their own remote storage media and databases. The sampled data is transmitted in real time to the remote storage media of the first subsystem that acts as a buffer to guarantee the remote and continuous reception of data. The data is transmitted from the first subsystem to the second subsystem where an immutable copy of the same is stored.

The at least one database of the first subsystem is controlled by an administrator node (hereinafter referred to as “secondary database”). Storage in this remote subsystem is architecturally decentralized (distributed).

The at least one database of the second remote storage subsystem (hereinafter referred to as “main database”) may be politically centralized (partially or fully) or decentralized. Preferably, the main database is politically decentralized. Storage in this remote subsystem is architecturally decentralized (distributed).

Accessibility to such databases for the generation of new records, consultation and correction thereof is executed by a governance system with which users interact, which may be partially or totally centralized, or decentralized.

The at least one primary or secondary database may be composed of one or more types of databases such as a relational database, a non-relational (NoSQL) database, a time series database, etc.

The record of the evolution over time of the variables that allow to describe and reconstruct an anesthesiological procedure is generated via the sampling and storage of a succession of measurements of these variables in a continuous fashion or by discrete time intervals known as “Time Series”. For a better interpretation of the recorded time series, supplementary data which additionally describes the anesthesiological procedure and events may be also added into the records.

The collected data constitutes a file that comprises one or more of the following:
a. Data on the patient 100 and/or on the procedure to be performed, entered by the anesthesiologist 118a, 118b prior to the anesthesiological procedure;
b. Geolocation;
c. Synchronization with Coordinated Universal Time;
d. Authentication in the at least one computing device 114 and in the at least one portable device 116;
e. Data from the sensors 110 connected to the patient 100 (sensor identifications, sampled values, limit thresholds, alarms triggered, sampling errors, etc.);
f. Connection quality and strength between each portable device 116 and the at least one computing device 114 and/or the at least one first data acquisition device 112;
g. Motion sensor data from each portable device 116;
h. Identification data of each manipulation with the at least one computing device 114 together with the data of whether there was close proximity interaction between at least one portable device 116 and the at least one computing device 114 at that time, including the identification of the portable device 116 if that was the case;
i. Internet connection quality data via Wi-Fi or mobile network;
j. Data of system internal errors, devices malfunction or communications issues;
k. Data from environmental sensors;
l. Checklists;
m. Supplementary data added manually during the procedure, including multimedia files sent from other compatible and user authorized devices;
n. Dynamic code 800 projected for video synchronization; and
o. Device battery level data.

In preferred embodiments, at least a file related to an anesthesiological procedure comprises at least the aforementioned data types a) to j). In even more preferred embodiments, the at least one file comprises all of the aforementioned data types.

The files of the same patient 100 are linked to each other forming an anesthesiological history of the patient 100.

Preferably, each history is identified with an alphanumeric code. Optionally, that alphanumeric code is generated by a hash function.

In embodiments, the patient 100 may provide user identification to an anesthesiologist 118a, 118b by providing the alphanumeric code and a personal password.

In an embodiment, storage is done by means of cloud computing where the storage media are located in multiple nodes in different geographical locations and hosted on the Internet. In addition, multiple copies of the records may be sent to be stored on the storage media of the different nodes.

A database management system is employed to build and manage the at least one database. By means of the database management system, it is possible to create application programs, store the data and easily retrieve it. However, these are defined in the database as unalterable and not removable, and correction and/or incorporations of new data, as long as they are allowed, are recorded preserving the original data and allowing the consultation of the history of authenticated corrections and incorporations. In this sense, allowing corrections and incorporations aims at making up for deficiencies or adding data, respectively, but in no way altering the original data that will continue to be part of the record.

Preferably, this database administration system belongs to an administrator node.

The employment of blockchain technology to perform the storage and/or recording the storage addresses is contemplated.

Preferably, the data is encrypted and distributed over the network to the remote storage system.

Furthermore, in embodiments of this invention, the system is configured to automatically fragment and/or reassemble the data. The encrypted data is partitioned and multiple copies of each partition are generated. These data partitions may be stored in different and separate locations.

In preferred embodiments the data collected and verified by the administrator node is stored in a main database. Additionally, the addresses of each data partition may be recorded in a blockchain along with data such as the identification of the record, user identification, geolocation, time schedule and hash of its content. In this way, an immutable record may be generated in a block of a blockchain that may function as “proof of existence” and may allow the reconstruction of the contents of the record and the verification of its indemnity.

In embodiments of the present invention, data collected during an anesthesiological procedure is transmitted in real time from the at least one computing device 114 to a remote storage system, governed by a partially centralized administrator node. The administrator node, among other functions, acts as a certifying intermediary, verifying the users and the data collected before sending them to the at least one main database, so as to ensure their quality and the protocols employed. When the anesthesiological procedure is finished, and therefore also the data transmission, the data belonging to an anesthesiological record is integrated into a file, which is hashed, encrypted and sent to at least one main database, without central authority. Preferably, this data is stored both in at least one secondary database, controlled by the partially centralized administrator node, and in the at least one main database.

Subsequently, the administrator node stores an alphanumeric code in a block of a blockchain that may contain data such as the identification and version of the anesthesiological record, identification of the anesthesiologist who generated the record, hash of the record's content, geolocation, UTC time and addresses in where the file containing the record is stored in the at least one main database. This registry on the blockchain is preferably executed with the lowest possible latency since the anesthesia record is stored in the main database. In this way, from the timestamp of a block on the blockchain, it is possible to determine the latency from the data sampling until its storage in the main database.

The alphanumeric code is also sent to the computing device 114 together with the identification of the blockchain and the specific block identification where it was stored, serving as confirmation and proof that the anesthesia record has been stored in the at least one main database.

The administrator node saves the key to decrypt the file containing the anesthesiological record.

It is possible to keep a copy of the records in the at least one secondary database to be easily accessed for research, data analytics and statistics.

In preferred embodiments, the recording system of the present invention additionally comprises a module for executing queries from the administrator node capable of detecting the existence of an anesthesiological history of the patient 100 and retrieving it so that the anesthesiologist 118a, 118b is able to access it.

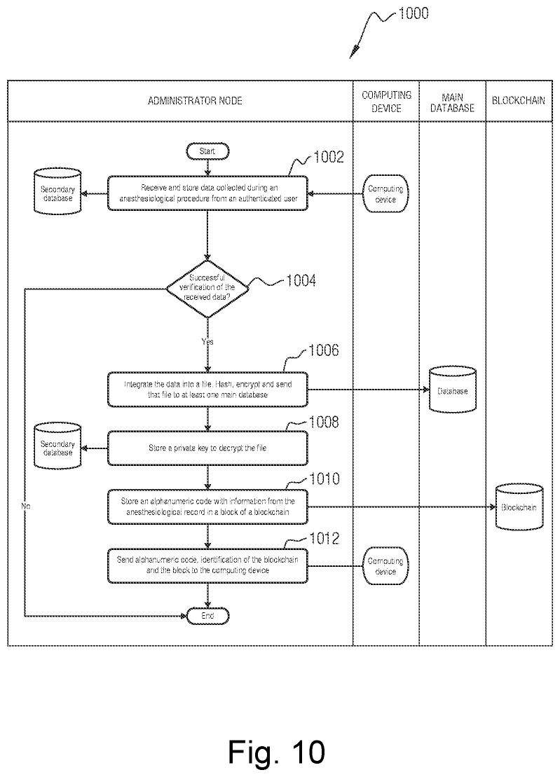

FIG. 10 shows a flowchart depicting a method 1000 for safeguarding a record of an anesthesiology procedure in accordance with one embodiment of the present invention.

Method 1000 begins with step 1002. In step 1002, data collected during an anesthesiological procedure is received from an authenticated user from a computing device 114 and stored in a secondary database. In step 1004, verification of that received data is performed. If the verification is successful, the operation proceeds to step 1006. Otherwise, the method ends.

In step 1006, the data is integrated into a file, which is hashed, encrypted, and sent to the at least one main database.

In step 1008, a private key to decrypt the file is stored in the secondary database.

In step 1010, an alphanumeric code with information from the anesthesiological record is stored in a block of a blockchain. Subsequently, the operation proceeds to step 1012 where that alphanumeric code is sent to the computing device 114 together with the identification of the blockchain and the block.

The information stored in the system may be retrieved by those registered users who are granted permission.

Patients 100 may retrieve and access their anesthesiological history (comprising their previous records) by an specific authentication code. The anesthesiological history contains specific information concerning the anesthesiological field. These records are useful for consultation in pre-anesthetic interviews for future interventions anywhere in the world. Unlike a general medical history, the anesthesiological history comprises specific information concerning the patient such as physiological responses to medications, medical history details, history of adverse effects, previous surgery interventions, medical treatments, previous anesthesia procedures and events, airway management details, among other information which may be useful in surgical or emergency procedures.

The administrator node may be accessed via the Internet by means of a platform from a computing device, preferably from the at least one computing device 114 of the recording system of the present invention. To consult a previous record it is necessary to be an authorized user and, therefore, to log in.

In preferred modalities, upon a request for access to an anesthesiological record made from a computing device 114 of the system, the administrator node verifies that this request comes from an authenticated user on the computing device 114 with sufficient permissions to access the record. After confirming that the request comes from an authorized user, the administrator node controls that the request contains the alphanumeric code of the record, verifies the existence of the code in the blockchain and checks the addresses of the file that contains the record.

Once the addresses have been verified, the administrator node downloads the record and decrypts it with the private key. Additionally, it verifies that the hash is correct, and thus verifies the identity and indemnity of the downloaded record. If verified, the user is granted access to the record from the requesting computing device 114.

In other words, to reconstruct a file containing the record of an anesthesiological procedure, the following is necessary:

a private key to authorize such reconstruction via the collection and decryption of files granted by the administrator node; and

the list of addresses where each of the record partitions are stored.

Thus, once the data has been transmitted, it becomes immutable and may only be accessed through the system that has the private keys.

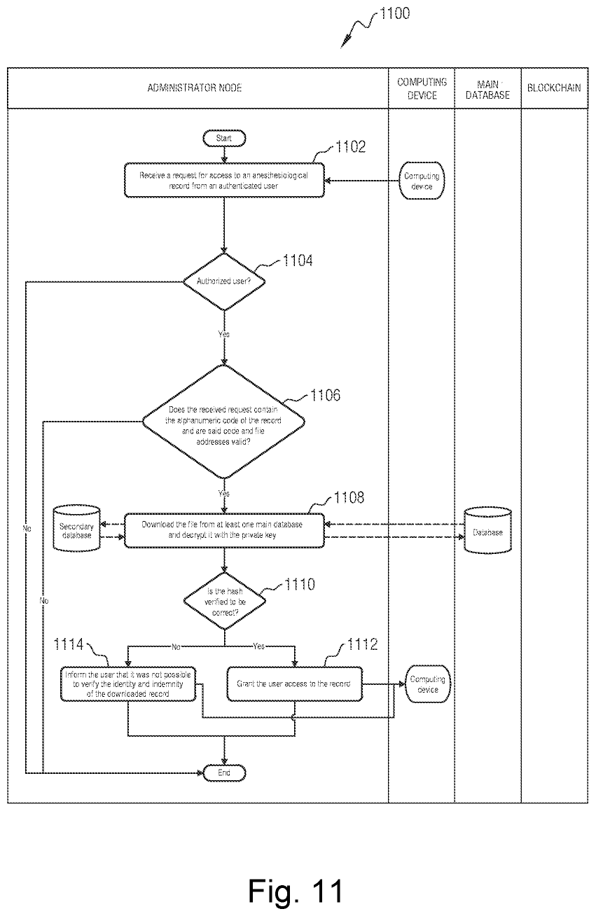

FIG. 11 shows a flowchart depicting a method 1100 for accessing a record of an anesthesiology procedure in accordance with one embodiment of the present invention.

Method 1100 begins with step 1102. In step 1102, a request for access to an anesthesiological record is received from an authenticated user from a computing device 114. In step 1104, permission verification is performed. If the requesting user is found to be authorized, the operation proceeds to step 1106. Otherwise, the method ends.

In step 1106, it is verified whether the received request contains the alphanumeric code of the record, and the existence of that code in the blockchain and the addresses of the file that contains the record are checked. If those checks are successful, the operation proceeds to step 1108. Otherwise, the method ends.

In step 1108, the file is downloaded from the main database, which is decrypted with the private key. The hash is then verified to be correct in step 1110. If the hash is correct, the operation proceeds to step 1112 where the user is granted access to the record. If the hash is not correct, the user is informed, in step 1114, that it was not possible to verify the identity and indemnity of the downloaded record.

In embodiments, once a record has been retrieved, when the user intends to make a correction or add new data to it, he enters the new data to the system via the computing device 114. The administrator node may then integrate the previous record data with the new data and generate a new file which is hashed, encrypted and sent to the at least one main database. Alternatively, the administrator node may create a new file containing only the new record data, hashing it, encrypting it, and sending it to the at least one main database.

Just as when a new record is sent to the at least one main database, an alphanumeric code related to the record is generated and subsequently stored in the blockchain. In the event that the new stored file only contains the new data, it may be specified in the new code that this file is the latest of the complete file set. All concatenations that are necessary to also download the previous versions so as to rebuild the complete anesthesia record may be referred in the files and in the blockchain.

Finally, the administrator node sends that alphanumeric code to the computing device 114 together with the identification of the blockchain and the block identification where it is stored.

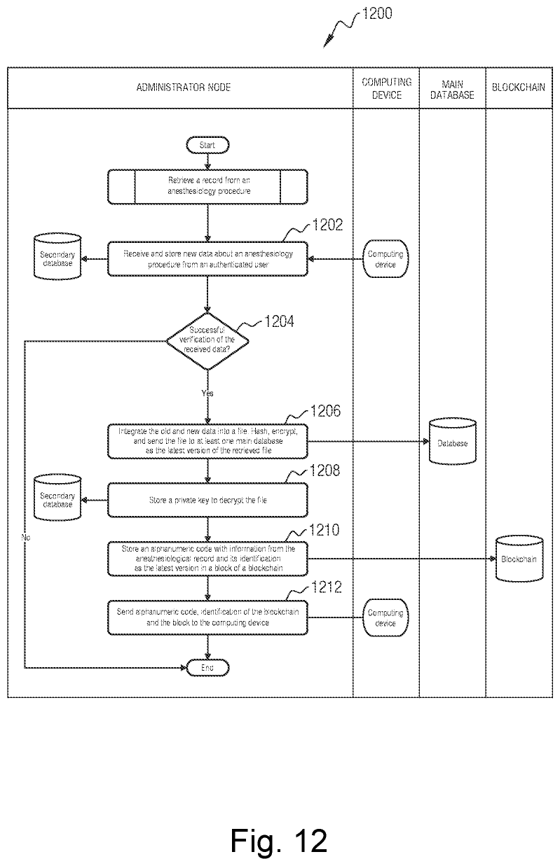

FIG. 12 shows a flowchart depicting a method 1200 for adding a correction or new data to an anesthesiological procedure record in accordance with one embodiment of the present invention.

Method 1200 begins with step 1202 after retrieving a record from an anesthesiology procedure. At step 1202, new data about an anesthesiology record is received from an authenticated user from a computing device 114 and stored in a secondary database. The addition of new data may be done with the intention of adding new information to the record so as to complete it, or correct previous data included in the record. In any case, the previous data does not undergo any modification, but the new data is added to them. In step 1204, verification of the received data is performed. If the verification is successful, the operation proceeds to step 1206. Otherwise, the method ends.

In step 1206, the old and new data are integrated into a file, which is hashed, encrypted, and sent to the at least one main database as the latest version of the retrieved file.

In step 1208, a private key to decrypt the file is stored in the secondary database.

In step 1210, an alphanumeric code with information from the anesthesiological record and its identification as the latest version is generated and stored in a block of a blockchain. Subsequently, the operation proceeds to step 1212 where that alphanumeric code is sent to the computing device 114 together with the identification of the blockchain and the block.

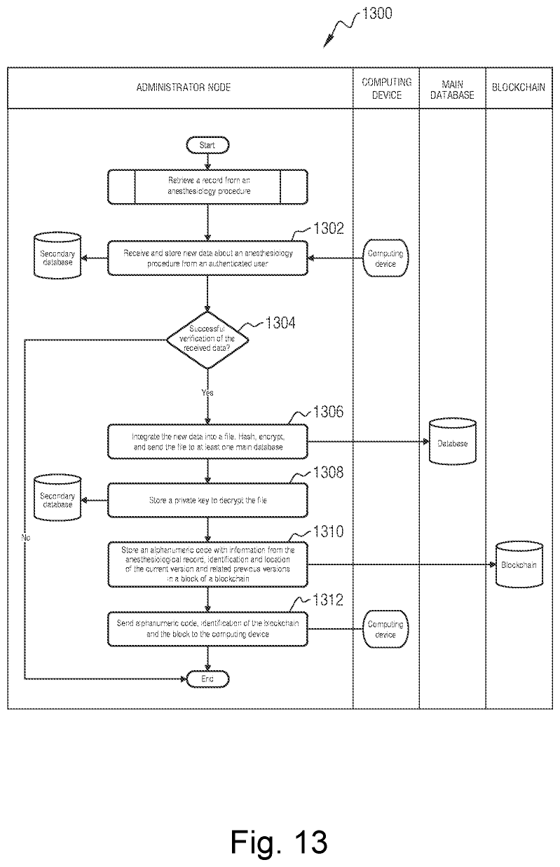

FIG. 13 shows a flowchart depicting a method 1300 for adding a correction or new data to an anesthesiological procedure record in accordance with another embodiment of the present invention.

Method 1300 begins with step 1302 after retrieving a record from an anesthesiology procedure. In step 1302, new data about an anesthesiology record is received from an authenticated user from a computing device 114 and stored in a secondary database.

In step 1304, verification of the received data is performed. If the verification is successful, the operation proceeds to step 1306. Otherwise, the method ends.

In step 1306, the new data is integrated into a file, which is hashed, encrypted, and sent to the at least one main database as the latest file of the complete file set.

In step 1308, a private key to decrypt the file is stored in the secondary database.

In step 1310, an alphanumeric code with information from the anesthesiological record, identification and location of the current version and related previous versions is stored in a block of a blockchain. Subsequently, the operation proceeds to step 1312 where the alphanumeric code is sent to the computing device 114 along with the identification of the blockchain and the block.

Requests, accesses and corrections/entry of new data are registered at least in the administrator node.

User authentication data is also stored as a security method to ensure that no one goes through the records unnecessarily in an attempt to correlate any record with a particular patient to disclose their information.

In embodiments, a special platform with selected content for the general population may be accessed without having to log in. On this platform, relevant statistical information for the community may be consulted.

The system may further comprise a data analysis module in the administrator node configured to generate statistics, which may be accessed, for example, via the at least one computing device 114. In a preferred embodiment, the analysis module is further configured to access previously generated statistical data and make it available in real time for contrasting it with the sampled data from the patient during the anesthesiological procedure. This information may be useful to interpret the current state of the patient 100 by comparing it with metrics of similar scenarios sampled by the users' community.

The system may also generate a universal personal professional log. Each anesthesiologist 118a, 118b registered in the system may access their professional log of anesthesiological procedures by authentication. Such logs may include, anesthesia records and statistical information such as types of procedures performed, locations where they were performed, schedule, duration, performance and quality of surveillance, completeness and accuracy of registered data, personal statistics and, in the case of anesthesiologist in training; data regarding the measurements of their supervised activities. By this way, the system aims to provide a globally interoperable professional log that overcomes the format variability and incompatibilities between current electronic and paper anesthesia records.

The anesthesiologists in training 118b, may access their professional log by authentication, which may include, the number and type of procedures performed and supervised by an anesthesiologist in charge 118a, the quality of surveillance that the patient 100 received from the anesthesiologist in training 118b and the quality of supervision that the the anesthesiologist in training 118b received from the anesthesiologist in charge 118a.

Furthermore, the proposed system may generate a rigorous database which may be available for scientific research, pharmacovigilance, adverse events reporting, optimization of patient safety protocols, performance analysis, quality improvement, costs evaluation and the like.

In another embodiment of the invention, the system includes a record classifier module in the administrator node capable of classifying the records of anesthesiological procedures by means of a comprehensive quality scale based on the completeness and accuracy of the record and the quality of surveillance. Completeness and accuracy are functions of the quality and quantity of the sampled data and supplementary information incorporated manually into the system by the anesthesiologist 118a, 118b. Quality of surveillance can be estimated as a weighted function of the measurements of the professional's continuous presence within a safe distance range from the patient 100.

The highest quality records are expected to be embraced more frequently by researchers who require scientific data, generating an incentive for anesthesiologists 118a, 118b to maximize the completeness and quality of their anesthetic records for scientific cooperation.

Anesthesiologists 118a, 118b may keep track of their statistics and may count on system certifications for records that are selected for research due to their scientific quality.

In preferred embodiments, each record is authenticated by the at least one anesthesiologist 118a, 118b involved in the procedure. The authentication signature and user identification may take the format of an alphanumeric code so as to preserve the anonymity of the professional. If a professional needs to contact or verify an anesthesiologist who generated a specific record, they may do so directly with their alphanumeric user or they may request the professional's identification to the administrator node. After direct approval or by consensus, the administrator node enables a second layer where the team of professionals 118a, 118b involved in the procedure may be identified if necessary. A record of identification requests may be generated.

In the context of the present invention, the term “real-time data transmission” should be interpreted as the transmission of data with very low to minimal latency, so that the data, once being sampled, remains for the shortest possible time out of the remote storage system and therefore vulnerable. Latency must be understood as the accumulated delay from one end to the other, that is, from the moment data is sampled on the source site until it is stored in the destination database. For the purposes of this invention, a preferable latency is less than or equal to the time it takes for an anesthesiologist 118a, 118b to observe signals, integrate them, interpret the scenario, and decide to act accordingly.

Therefore, the system of the present invention is capable of transmitting and storing data in immediate or almost immediate response to its generation, maximizing its integrity and security.

In possible embodiments of the present invention, the data is stored in a storage medium of the at least one computing device 114 that acts as a buffer in the event the real-time transmission of data is interrupted, for example, due to a decrease in quality or strength of Internet connection. By this way the sampled data that is unable to be transmitted in real time during that period is subsequently transmitted from the at least one computing device 114 to the remote storage system as soon as the connections are reestablished. The data may be labeled based on the latency of its transmission to the remote storage system. The backup stored in the at least one computing device 114 may be automatically deleted or be overwritten after confirming that it is properly received and stored in the at least one main database.

To clarify, in such embodiments, the system may prioritize to transmit the data in real time, but when this is not possible, the data may be transmitted in a deferred way from the at least one computing device 114 to the remote storage system. Transmitted data may be labelled with respect to latency of transmission with respect to sampling time.

The data is preferably transmitted as streams or sequences of data packets with a preferably minimal interval between packet egress, although other forms of transmission are contemplated. Each data packet may comprise a timestamp.

In other embodiments, the administrator node has a consensus system for the authorization of access to network information (for research purposes, for instance), where an electronic query is opened to users so that they decide to accept or deny access (always preserving the anonymity of the patients) to information by interested third parties. Such consensus includes an authentication and registration of the users who participate in the voting process.

In some embodiments, the administrator node counts with a consensus system by which users may be invited to review anonymous data on anesthesia records that require audit, interpretation or professional peer review. Each user may express their review and opinion so as to establish a feasible reconstruction of the facts among all the users which cooperated.

Preferably, auditing users must be authenticated, but the anonymity of the source of the reviewed records is always preserved.

In preferred embodiments, the system may count on a governance mechanism for decisions or verifications that need to be made by its user community. A consensus mechanism may be utilized, via a query system. Users may vote or be selected to vote, weighting their professional and scientific experience.

It is within the scope of the present invention to incorporate methods, tools and technologies known in the art aimed at protecting information and, therefore, computer systems, against any threat. Security measures comprise at least physical and logical security measures.

Physical security measures refer to mechanisms to prevent direct or unauthorized physical access to the system. They also protect the system from natural disasters or adverse environmental conditions. Three fundamental factors to consider are physical access to the system by unauthorized persons, physical damage by harmful agents or contingencies, and recovery measures in case of failure.

As an example, the types of controls that may be set include:
Control of environmental conditions such as temperature, humidity, dust, etc.
Prevention of catastrophes, that is, fires, power cuts, overloads, etc.
Surveillance, including cameras, guards, etc.
Contingency systems such as fire extinguishers, uninterruptible power supplies, voltage stabilizers, alternative ventilation sources, etc.
Recovery systems: backups, redundancy, geographically separated and protected alternative systems, etc.

Logical measures include protocols and/or policies to provide access to resources and information and the correct use thereof, as well as the distribution of responsibilities among users.

Among the types of logical controls that may be included in a security policy, the following may be highlighted, by way of example, but not limitation:
Establishment of an access control policy, including an identification and authentication system for authorized users and an information access control system.
Use of cryptography to protect data and communications.
Use of firewalls to protect a local area network connected to the Internet.
Definition of a backup policy.

Exemplary modes are detailed below of some types of sensors 110 capable of sampling physiological data of a patient 100 and of their interaction with electromechanical devices to which they are connected compatible with the system of the present invention. The following sensor examples and their details should not be construed as limiting the scope of the present invention.

Pulse Oximeter

Pulse oximetry is a technology that indirectly monitors the oxygen saturation of a patient's blood producing a photoplethysmogram that may be further processed into other measurements. It consists of the application of specific wavelength photodiodes that emit light capable of passing through tissues, generally the fingers, and is detected by a photodetector on their other side. The difference between the emitted and received electromagnetic spectrum generates a signal from which the percentage of saturation of the patient's hemoglobin may be indirectly inferred. The signal may be depicted by a Cartesian axis where time is represented on the abscissa axis and the pulse amplitude is represented on the ordinate axis. The percentage of hemoglobin saturation is represented by a number from 0 to 100.

Its utility consists of measuring hemoglobin saturation, heart rate, arrhythmias, and quality of tissue perfusion.

Sometimes two pulse oximeters are employed so as to establish a relationship between two different sites in the body that may have perfusion differences during surgical procedures such as correction of congenital heart disease, repair of aortic aneurysms, etc.

Electrocardiograph

The electrocardiograph comprises a system with a plurality of cables, for example, 3 to 12 cables, which are connected by means of adhesive electrodes over the patient's chest and on its sides. They are capable of measuring electrical potential differences generated by the heart and depict it by means of a continuous flow Cartesian graph in which time may be represented on the abscissa axis and the electrical potential on the ordinate axis. Thus, electrical complexes are generated representing cardiac activity between different topological axes determined by the position between electrodes.

Its utility consists of measuring heart rate, detecting cardiac tissue conduction abnormalities, arrhythmias, cardiac arrest, determining cardiac axis, detecting certain pathologies such as myocardial hypertrophy, cardiac tamponade, etc., detecting ventilatory rate (by measuring impedance variation between leads during inspiration and expiration of the patient).

Non-Invasive Blood Pressure Measurement System

The non-invasive blood pressure measurement system comprises an adaptable cuff s that is applied around the patient's arm, forearm, thigh or leg of the patient and closes with a hook-and-loop fastener. It is connected by a hollow hose to the data acquisition device that consists of a pressurizer motor and a mechanical transducer that transforms the pressure signal into an electrical signal.

So as to perform the measurement, an electric motor coupled to a valve that works as a pressure pump is activated, increasing the pressure in the cuff until the mechanical transducer stops measuring oscillations transmitted by the heartbeats. Once this point is reached, pressure is gradually released until a wave of pressure variation produced by the heartbeat is detected again. Once the optimum level of pressure difference in the wave has been reached, the mean pressure is calculated and, with this value, a formula is applied to estimate the systolic and diastolic pressure.

The measured data may be represented by three numbers representing the systolic, diastolic and mean blood pressures.

This system may be programmed to perform intermittent measurements according to a desired interval.

Its utility consists in measuring blood pressure in a non-invasive way.

Invasive Pressure Measurement System

Invasive pressure measurement systems consist of mechanical pressure transducers which are connected directly by a small non-compressible tubing to a catheter placed within the patient's vasculature.

The measurements are generally executed in the arteries of the arms, although they may be conducted in any vessel or space where direct pressure measurement is required (blood vessels, heart chambers, pleural, subarachnoid, epidural, abdominal spaces, etc.).

In embodiments where the system of the present invention is the only monitoring system, the mechanical pressure transducer is connected to the at least one first data acquisition device 112 so as to receive the captured signal directly. In other embodiments where the system is complementary to another monitoring system, placing a new catheter and transducer to obtain an exclusive signal for the system of the present invention would be undesirable as it would be redundantly invasive for the patient. In these cases a lateral signal capture system is preferably applied.

Taking advantage of the fact that mechanical pressure transducers are universal, their sampled signal is replicated by means of a system for copying the original signal that is sent to the anesthesia machine or monitor without disturbing it.

The mechanism may consist of coupling a special device between the cable of the anesthesia machine and the mechanical transducer. That part is connected to the at least one data acquisition device.

Since pressure sensors connected to a cable extender are employed in most current measurement systems, an accessory capable of being connected between them may be applied.

The principle of operation for most invasive pressure sensors is of the bridge type. From the deformation of a resistive membrane, a small output signal is obtained as a result of the imbalance of the resistive measurement bridge present in the sensor. This output signal is amplified in a proportion according to the range possessed by the equipment that is responsible for the sampling and digitization of the signal.

Most of the standard sensors have four wires in their connector, with two of them to power the sensor while the other two transmit the output signal.

Preferably, a bridge is provided between the sensor and the extension cable for these four lines, being totally transparent to the main monitor that is measuring the signal.

In some embodiments, the signal replication device does not have its own power supply, but employs the power provided by the multiparameter acquisition device that contains batteries. In this manner, ground problems between acquisition devices are avoided and the electrical isolation of the patient is maintained with respect to ground potential and any other potential referred to it that allows dangerous currents to flow through it.

Preferably, the signal to be measured is taken from the signal lines coming from the sensor and is amplified by means of an instrumentation amplifier with high input impedance, high rejection of CMRR (Common Mode Rejection Ratio) and low noise, which makes for a first stage of signal amplification with configurable gain. Due to its very high input impedance, it does not alter the original signal that continues its path to the main monitor. A second filtering stage is placed in cascade by means of a low-pass filter for the elimination of line and high-frequency noise in the signal, which, in addition, may meet the necessary requirements to act as an anti-aliasing filter for correct sampling and signal reconstruction in digital format. The data is sent to the acquisition device by means of three-pole wiring.

Examples of connectors that may be utilized are: BD connector, Abbot connector, B. Braun connector, Edward connector, Uath connector among others.

The invasive pressure signal may be depicted by a continuous flow Cartesian graph, with time represented on the abscissa axis and pressure on the ordinate axis. This graph consists of pressure waves of different characteristics depending on the place of sampling: blood vessels, spaces or cavities.

Its utility consists of measuring body pressures directly and continuously, allowing the estimation of several variables such as myocardial contractility, arrhythmias, intravascular volumes, etc.

Preferably, the system comprises three invasive pressure measurement systems so that several pressures from different body sites may be sampled simultaneously according to the needs of the procedures.

Electroencephalography Data Measurement System

The single- or multi-channel electroencephalography data measurement system for capturing and storing information on the patient's brain activity during the anesthetic procedure consists of several cables similar to electrocardiograph sensors, but measuring much smaller potential differences generated by brain activity, for example, of the frontal lobes.

The sensors are placed in different areas of the patient's head, depending on the procedure requirements. Sampled signals are filtered and amplified.

The hardware may comprise electrodes, an analog signal adaptation block, an analog-digital conversion block and a digitized signal storage and transmission system. These signals may be, in turn, depicted on the screen for real-time monitoring.

Its utility consists of recording states and depths of anesthesia, predicting anesthesia depth in order to avoid intraoperative awareness. It may be particularly useful for collecting data for science research.

Temperature Sensor

Temperature sensors consist of two wires that have a thermistor at the end. Via the electrical resistance generated by the material, the surrounding temperature may be determined. Preferably, two channels are available for measuring temperature at two body sites simultaneously (e.g. pharyngeal, rectal, or body surface temperature).

They are useful in prolonged surgeries or those that require extracorporeal circulation, since they are employed to measure differential temperature during cooling and rewarming so as to detect perfusion abnormalities.

Its utility consists of measuring internal temperature, body surface temperature, and differential temperature between different areas of the body.

Gas Composition Sensor

It consists of a set of sensors capable of measuring the concentration of different gases. Carbon dioxide and anesthetic gases may be measured via spectrophotometry by mainstream or sidestream system. Oxygen may be measured by galvanic sensors.

Preferably, the sensor is a mainstream gas (for carbon dioxide, and volatile anesthetics) composition sensor consisting of a hard sanitizable tubular piece that fits between the distal region of the corrugated tubing of a ventilator. This piece has two glass windows facing each other in which an array of diodes and spectrophotometric sensors are attached on opposite sides. Detection is done by specific electromagnetic frequencies that are emitted and absorbed by specific gases.

This device ideally works coupled to gas pressure, flow and volume sensors in a same unit.

In another embodiment, the gas sensor may be of the sidestream system type that consists of a small tube with negative pressure that absorbs and transports the gases from the tube to the sensors located distally from it.

Concentrations of carbon dioxide, and various anesthetic gases, including sevoflurane, isoflurane, desflurane, enflurane, may be measured in real time.

The measurement may be depicted on the screen as a Cartesian graph of continuous flow specific for each gas, in which time is represented on the abscissa axis and concentrations on the ordinate axis.

Its utility consists of measuring:
Carbon dioxide concentration exhaled and inspired by the patient. This data is important to estimate, among other things, correct intubation, airway pressurization, cardiac output, detection of adverse events such as malignant hyperthermia, need for replacement of soda lime of the anesthesia machine, etc.
Oxygen concentration is utilized to continuously visualize the oxygen mixture that is administered to the patient, and for avoiding hypoxic mixtures. In specific patients subtle oxygen concentration variations may generate important hemodynamic and ventilatory effects.
Volatile Anesthetics concentration is measured continuously so as to optimize the administered doses and detect variations that could lead to overdosing, for example, due to distraction, or underdosing, due, for instance, to total inadvertent consumption of the inhalation agent in the anesthesia machine.

Airway Pressure, Flow and Volume Sensor

The airway pressure, flow and volume sensor consists of a hard, sanitizable tubular part that is preferably connected together with the “mainstream gas composition sensor”. It has a passageway in its interior towards two small membranes of different diameter that function as mechanical pressure transducers. This array of sensors may measure and calculate three parameters in the airway: pressure, flow, and volume.

The mechanisms by which these parameters are measured are explained below:
Pressure: it is measured directly and continuously via either of the two pressure sensors.
Flow: it is calculated by the relationship between the measurements between both pressure sensors of different diameter. This calculation and its representation may be performed continuously.
Volume: it is calculated using the above-mentioned continuous flow quantification and the measured time during which the flow and its variation are recorded.

The three parameters may be depicted on the screen in various ways:
a) independently from each other as a continuous flow Cartesian graph in which time is represented on the abscissa axis and pressure, flow or volume on the ordinate axis.
b) two of these parameters may be represented in relation to each other where one is represented on the abscissa axis and the other on the ordinate axis, generating a regenerative graph with each ventilatory cycle called a “loop”. Such loops are generated by representing the relation between pressure and volume, pressure and flow, or flow and volume. From these loops, additional parameters such as “lung compliance” may be calculated.

Its utility consists in the continuous measurement of the physical characteristics of the patient's pulmonary system, including lungs' efficiency to exchange gases and its variations in compliance. Sudden changes to these parameters may be useful to detect possible adverse effects such as kinking of the endotracheal tube, disconnections, alterations in the lung parenchyma, ventilator failures, etc.

Preferably, the mainstream gas composition sensor along with the airway volume, flow and pressure sensor are coupled together and share a wired connection with the acquisition device. They work as a single unit, and the integration of their sensors allows the calculation and representation of other data such as Volumetric capnography. This is the integration of the flow and volume calculation together with the concentration of exhaled carbon dioxide. This data is depicted in various Cartesian graphs and data such as mass of exhaled carbon dioxide, alveolar dead space, and anatomical dead space may be calculated.

Its utility consists in:
Measuring the total amount of carbon dioxide expelled by exhalation, which allows for a precise estimation of blood flow and gas exchange efficiency in the lungs.
Measuring dead space volumes, which are volumes of gases in the ventilator circuit and the patient's lung that do not participate in gas exchange. Intermittent measurement is done to visualize gas exchange trends which in turn are useful for evaluating the performance of gas exchange in the lungs during different maneuvers especially in thorax surgeries.

Near Infrared Spectrometry (NIRS) Data Measurement System

The near infrared spectrometry measurement system consists of four channels that have a spectrophotometric emitter and sensor in their distal region. The principle of operation is similar to that of the pulse oximeter, but differs in that the emitter and sensor are on the same surface that adhere to the patient's skin in the head or splanchnic region. In this way, the emitted light is detected by the photoreceptors after passing through and being reflected by deeper tissues.

Another difference is that light-emitting diodes emit a specific electromagnetic frequency so as to measure oxygen saturation deep within tissues, hence the name “near infrared”.

Preferably, four of these sensors are employed: two to measure bilateral frontal encephalic saturation and the other two to measure bilateral splanchnic saturation.

The data may be displayed on the screen as specific numbers for each channel from 0 to 100 that represent the saturation of the blood circulating in the tissues. It may also be depicted as a “trend” in a continuous displacement Cartesian graph where time is represented on the abscissa axis and the percentage of saturation of each channel on the ordinate axis.

Its utility consists in continuously measuring oxygen saturation levels for each body region. This data is utilized to evaluate the trends and modifications that occur with the different therapeutic strategies and to compare the differences in perfusion between the right/left or superior/inferior hemibodies.

The configurations disclosed in the present invention reduce the risk of data tamper, destruction, and unauthorized access. The system and method of the present invention makes the recording and reconstruction of anesthesiological procedures immutable, more reliable and efficient, incentivizing the avoidance of any type of action that directly or indirectly may reduce patient safety during those procedures. The implementation of the present invention ensures efficacy and immutability of the anesthesiological recording process and promotes the generation of scientific information. Furthermore, it is applicable and reproducible in any anesthesiological procedure, granting the same desired effect.

The records generated by the system and method of the present invention are resistant to malicious modifications and allow the reconstruction and analysis of anesthesiological procedures, providing total transparency towards the controlled system, that is, the patient, and contributing to improve the performance of the controller, that is, the anesthesiologist. Rigorous data records may be available for scientific research and auditing purposes.

So as to ensure patient safety, the ideal registration system of the present invention may have, according to preferred embodiments, the following characteristics:
Synchronicity: data is sampled and recorded simultaneously with the occurrence of the measurements, and not in an anachronistic way as may happen in paper anesthesia records.
Real-time transmission: data is transmitted and stored in the remote storage system simultaneously with the sampling process or with the lowest possible latency.
Architecturally Decentralized, Encrypted and Redundant Storage: data is encrypted, partitioned and stored with a maximum global distribution, generating multiple copies of each partition.
Authentication: it has a signature or authentication mechanism that allows certifying that the data has been generated by whomever claims to have generated it.
Traceability: it has mechanisms that allow identifying the source of the generator of the records, including user, time and the geolocation.
Autonomy-Independence: it is independent from any other company that produces monitoring systems or anesthesia machines, so as to function autonomously and impartially, guaranteeing the absence of conflicts of interest.
Identifiable technology: the sensors employed are electronically identifiable so as to allow comparability of the sampled data as well as to audit sensors' technology and performance. Each sensor identification is transmitted along with the record.
Transmission of raw data/open source: data is transmitted and recorded without processing or with open source processing, so as to provide transparency and availability to the community about the sampling and post-processing of the information.
Automaticity—Non-Distractor: the sampling and generation of the anesthesia record occurs automatically, avoiding the distractions of intermittent manual registration of measurements.
Redundancy: data is sampled independently as a backup dataset from the original monitors of the operating room or anesthesia machine.
Predictive Analytics Compatibility: sampled data streams may be compared in real time with statistical data stored in the system database.
Accessibility and Availability: anesthesia records may be instantly accessed anywhere in the world.
Confidentiality: patient identity is protected at all times. No personal information can be leaked from the records as no personal identity information is incorporated in them. No record can be linked to a particular patient without their will and permission as the patient theirself has the alphanumeric code for identifying their own records.
Integrity—Unmodifiable: collected data is immutable from the sampling and transmission process to storage and retrieval.
Temporal-spatial relation registry between the controller system and the controlled system: the sampled, transmitted and stored dataset, includes dynamic measurements of the presence and activity of anesthesiologists within the safety range of their patient.
Temporal-spatial relation registry between the professional in charge and professionals in training: the sampled, transmitted and stored dataset includes dynamic measurements of the presence and activity of the trainer anesthesiologist and the anesthesiologist in training.
Blockchain Proof of Existence Registry: the addresses for the reconstruction of each anesthesia record, such as its identification by geolocation, time schedule and user authentication, are registered on a blockchain.
Blockchain Record History Registry: Accesses, amendments and version history of anesthesia records are registered on a blockchain.
Programmable Blockchain Compatibility: record system is able to operate with blockchains' smart contracts.
Supplementary Data Incorporation Compatibility: users may add supplementary data during the anesthesia record or in a deferred way such as in extra operative consultations.
Supplementary Data Synchronization: sampled data may be synchronized with third parties devices information such as video files.
Specific for anesthesiology: the system is designed exclusively for the use by anesthesia professionals.
Customizable Information Display: sampled data may be displayed on a screen according to the user's visualization preferences. A toggleable standard screen configuration is available for quick interpretation between colleagues in emergency scenarios.
Customizable Tools Compatibility: data analysis software tools may be installed according to users' preferences.
Portable Device Programmable Alert System: In addition to the system standard safety alerts, personal warning systems may be implemented on the portable devices according to the users' needs and preferences.

Optimal anesthesia record systems and methods are those that best allow reconstructing and interpreting an anesthesiological procedure by sampling and storing a complete and rigorous datasets. Authenticated Real Time Transmitted Time Series may be an appropriate solution as it can be integrated with blockchain technology and decentralized storage systems. Strategies such as timestamping, geolocation, spatio temporal measurements of system components, manual data incorporation and third party devices data integration will provide an optimal anesthesia record architecture.

By “complete” it should be understood to include the greatest amount of measurements and supplementary data necessary to perform a complete reconstruction of the facts.

By “exact” it should be understood that the measurements reflect the real values, are geolocated, synchronized, authenticated and traceable.

The completeness and accuracy of the set of measurements and data may be ensured if the record is safeguarded quickly and remotely from the site where the measurements originate.

With this invention it is possible to:
incentivize optimal patient surveillance;
generate an immutable anesthesiological record with scientific rigor (a continuous, real and unadulterable/immutable record is guaranteed);
assure global record accessibility and availability;
generate a global interoperable verification system for certifying supervised training, professional experience and personal performance;
generate a global quality standard that facilitates professional exchanges internationally;
generate reliable scientific data at a global level;
employ real-time metrics to optimize intraoperative anesthesiological performance;
incentivize scientific research;
encourage high-quality professional training;
provide a second monitoring system as a backup that increases the availability of patient information;
generate evidence to costs and professional payment determine minimum fees consistent with the complexity of the procedures; and
generate synchronized video recordings for the scientific record of surgeries.

The anesthesiological records generated by the system and method proposed in this invention contain specialized data for the anesthesiologist and emergentologist, of verified quality and with an easily interpretable universal format, allowing them to make better decisions. This may result inefficient with the existing digital medical records in the art as they have heterogeneous and redundant information about the patient without content quality verification mechanisms.

It is desirable for the optimization of control systems to count on measurement recording technologies which not only provide immutable datasets on systems self performance but also on systems' components spatio-temporal relation dynamics.

The proposed recording system architecture aims to generate and secure scientific rigorous measurements for anesthesiology modelization.

By continuously sampling evidence, quality improvement and patient safety maximization are encouraged.

Better models may allow anesthesiologists to make better decisions.

Throughout this specification the term “patient” is employed so as to refer to a subject who receives or is able to receive a medical service via an anesthesiological procedure.

Furthermore, the term “connected”, unless otherwise indicated, should be broadly interpreted as comprising: “connectable” (capable of being connected), “directly or indirectly connected”, “electrically connected”, “communicatively connected”, “attachable”, “directly or indirectly attachable”, “communicatively attachable”, among others.

The terms “capturing”, “sampling” and “collecting” are used interchangeably throughout this specification.

It is to be understood that the present invention is not limited to the embodiments exactly described, but various changes and modifications may be made without departing from the spirit of the scope of the present invention. Additionally, although the term “step” may be employed here to connote different elements of the methods employed, the term should not be construed to imply any particular order among the various steps disclosed herein unless and except when the order of individual steps is explicitly described. All these embodiments must be considered within the scope of protection of the claims that follow.

1. A recording system for anesthesiological procedures, comprising:
at least one first data acquisition device connectable to one or more sensors capable of capturing physiological data of a patient and of their interaction with electromechanical devices to which the patient is connected;
at least one electronic device portable by at least one anesthesiologist; and
at least one computing device connected, or integrated, to the at least one first data acquisition device and connected to the at least one portable device,
wherein the at least one portable device is configured to generate, via interaction with the at least one computing device and/or the at least one first data acquisition device, data related to the temporal-spatial relationship between the at least one anesthesiologist and the patient and, therefore, to the quality of surveillance provided by the at least one anesthesiologist to the patient, and
wherein the at least one computing device is configured to receive and transmit the collected data in real time to a remote storage system wherein an immutable copy of such data is stored.

2. The system of claim 1, additionally comprising at least one second data acquisition device connectable to one or more sensors capable of measuring environmental conditions at the site where the anesthesiological procedure is executed, the at least one second data acquisition device being connected to, or integrated into, the at least one computing device and/or the at least one first data acquisition device.

3. The system of claim 1, wherein each computing, portable and data acquisition device has a passive or active electronic system for identifying the model and serial number that are transmitted to the remote storage system, as well as those of the sensors to which each data acquisition device is connected.

4. The system of claim 1, wherein the at least one computing device is connected to the at least one first data acquisition device and with the at least one portable device through a first wireless connection.

5. The system of claim 4, wherein said first connection is a radio frequency connection.

6. The system of claim 1, wherein the at least one computing device is capable of continuously transmitting data on the quality of the connections to the remote storage system.

7. The system of claim 1, wherein the at least one first data acquisition device has the capability to connect to sensors capable of capturing physiological data of a patient and of their interaction with electromechanical devices to which the patient may be connected selected from the group that comprises pulse oximeters, electrocardiographs, non-invasive blood pressure measurement systems, invasive pressure measurement systems, electroencephalography data measurement systems, temperature sensors, gas composition sensors (carbon dioxide, oxygen and volatile anesthetics), airway pressure, flow and volume sensors, and Near Infrared Spectrometry (NIRS) measurement systems.

8. The system of claim 1, wherein the at least one first data acquisition device has the capability to connect to sensors capable of capturing physiological data of a patient that are independent of any other monitoring system.

9. The system of claim 2, wherein the at least one second data acquisition device has the capability to connect to sensors capable of measuring environmental conditions at the site where the anesthesiological procedure is executed selected from the group comprising temperature, humidity, and pressure.

10. The system of claim 1, wherein the at least one computing device comprises at least one input device capable of allowing the at least one anesthesiologist to enter supplementary data during the anesthesiological procedure.

11. The system of claim 1, wherein the at least one computing device comprises a display screen capable of rendering the received data in real time.

12. The system of claim 1, wherein the at least one computing device further comprises an authentication system.

13. The system of claim 12, wherein the authentication system comprises a fingerprint reader.

14. The system of claim 4, wherein the at least one portable device and the at least one computing device are additionally connected by means of a second wireless connection, which is a short-range connection, when they are at a distance equal to or less than a value considered suitable for user manipulation, wherein data on the identity of said at least one portable device in close proximity to the at least one computing device is transmitted through the second wireless connection to the at least one computing device allowing to associate a manipulation of the at least one computing device with an anesthesiologist if at the time of said manipulation at least one portable device was connected to it through the second wireless connection.

15. The system of claim 14, wherein said second wireless connection is a short-range radio frequency connection; the at least one portable device comprising a short-range RFID transponder capable of transmitting the identity of the at least one portable device associated with a single anesthesiologist; and the at least one computing device comprising a short-range RFID transmitter/receiver capable of capturing the RFID transponder signal of at least one portable device when the distance is equal to or less than a value considered suitable for user manipulation.

16. The system of claim 15, wherein the at least one computing device has some lockable functionalities and is further configured to unlock them when at least one portable device is identified at a distance equal to or less than a value considered suitable for user manipulation.

17. The system of claim 1, wherein the at least one computing device has the capability to connect to the Internet in order to receive information and transmit the collected data in real time to the remote storage system.

18. The system of claim 17, wherein the at least one computing device allows to determine, through the Internet connection, the geolocation and time synchronized with Coordinated Universal Time (UTC).

19. The system of claim 1, wherein the at least one portable device comprises at least one motion sensor capable of detecting presence or absence of movement signals from that portable device.

20. The system of claim 19, wherein the at least one motion sensor is an accelerometer.

21. The system of claim 1, wherein the at least one portable device further comprises an authentication system.

22. The system of claim 21, wherein the authentication system comprises a fingerprint reader.

23. The system of claim 1, wherein the at least one portable device further comprises at least one of: a plurality of buttons and a scrollable interface, configured to remotely control the at least one computing device.

24. The system of claim 1, wherein the at least one portable device further comprises a vibration system.

25. The system of claim 1, wherein at least one of the portable, computing and first data acquisition devices further comprises an audible alarm system.

26. The system of claim 1, wherein at least one of the portable, computing and first data acquisition devices further comprises a microphone incorporated into the device.

27. The system of claim 1, comprising a plurality of portable devices, one used by a main anesthesiologist and at least one other by a collaborating anesthesiologist or anesthesiologist in training, the portable device of the main anesthesiologist being in communication with each of the portable devices of collaborating anesthesiologists or anesthesiologists in training and, in turn, each portable device being in communication with at least one computing device.

28. The system of claim 1, wherein the at least one device portable by the at least one anesthesiologist is a wearable device.

29. The system of claim 28, wherein the at least one wearable device is an electronic bracelet.

30. The system of claim 28, wherein the wearable device is attachable to the body of the anesthesiologist through an opening and closing fastener provided with an electronic contact.

31. The system of claim 14, wherein:
the at least one portable device is connected via a first wireless connection to the at least one computing device and, optionally, also to the at least one first data acquisition device;
the at least one portable device comprises at least one motion sensor capable of detecting presence or absence of movement signals from said portable device; and
the at least one portable device and/or the at least one computing device comprise an authentication system.

32. The system of claim 31, wherein the data related to the temporal-spatial relationship between the at least one anesthesiologist and the patient and, therefore, to the quality of surveillance provided by the at least one anesthesiologist to the patient, generated by the interaction between the at least one portable device and the at least one computing device and/or the at least one first data acquisition device, comprise one or more of:
data on the proximity and/or distance between the at least one portable device and the at least one computing device and/or the at least one first data acquisition device and, therefore, between the anesthesiologist and their patient based on the intensity and/or quality of the signal of the first wireless connection between the devices;
data on the activity and/or inactivity of the anesthesiologist measured by means of the at least one motion sensor of the at least one portable device, detecting the presence or absence of movement;
authentication data of the anesthesiologist in the at least one computing device and/or in the at least one portable device; and
data on authenticated manipulations of the at least one computing device and/or of the at least one portable device.

33. The system of claim 32, wherein the at least one portable device and the at least one computing device are additionally connected by means of a second wireless connection, which is a short-range connection, when they are at a distance equal to or less than a value considered suitable for user manipulation and wherein the data related to the temporal-spatial relationship between the at least one anesthesiologist and the patient and, therefore, to the quality of surveillance provided by the at least one anesthesiologist to the patient, generated by the interaction between the at least one portable device and the at least one computing device and/or the at least one first data acquisition device, further comprises:
data on the identity of at least one portable device in close proximity to the at least one computing device allowing to associate a manipulation of the at least one computing device with an anesthesiologist if at the time of said manipulation at least one portable device was connected to it through the second short-range wireless connection.

34. The system of claim 1, further comprising a video synchronization device connected to the at least one computing device, wherein the synchronization device comprises an array of lasers that are projected alternately in the vicinity of an area of interest that is being recorded by any video camera, generating a dynamic and variable light code, said code being transmitted in real time to the computing device and, from there, to the remote storage system together with the other collected data, allowing the subsequent synchronization of the video with the recorded anesthesiological datasets.

35. The system of claim 1, further comprising a query execution module capable of detecting the existence of an anesthesiological history of the patient and retrieving it so that the anesthesiologist can access it.

36. The system of claim 1, further comprising a data analysis module with the capability to access statistical data in real time and compare it with the patient data that is being captured during the anesthesiological procedure.

37. The system of claim 1, further comprising a record classifier module capable of classifying records based on the completeness and quality of the data they include.

38. Device portable by at least one anesthesiologist applicable to the system of claim 1, comprising:
at least one first wireless communication system capable of connecting to at least one computing device and/or at least one first data acquisition device by means of a first wireless connection and of transmitting through it data on the proximity and/or distance between the portable device and the at least one computing device and/or the at least one first data acquisition device and, therefore, between the anesthesiologist and their patient based on the intensity and/or quality of the signal of the first wireless connection between the devices and data on the identity of the portable device;
at least one motion sensor capable of detecting movement signals from the anesthesiologist; and
at least one authentication system capable of verifying the identity of a user carrying the portable device in response to receiving an authentication request from the at least one computing device,
wherein the data on the activity and/or inactivity of the anesthesiologist measured by the at least one motion sensor and the authentication data is transmitted to the at least one computing device via the first wireless connection.

39. Method for recording anesthesiological procedures implementable in the system of claim 1, comprising the steps of:
receiving in the at least one computing device a login credential from at least one anesthesiologist who accesses the at least one computing device and, in response to the authentication of the anesthesiologist via an authentication system, establishing a connection between the at least one computing device, the at least one portable device and the at least one first data acquisition device:
generating at least one file to contain data on the anesthesiological procedure;
receiving in the at least one computing device preliminary data about the patient and/or about the procedure to be performed, via an input device connected to, or integrated into, it, and send that preliminary data to the remote storage system;
receiving confirmation from the at least one anesthesiologist, via authentication by the at least one computing device, that the anesthesiological procedure is about to begin;
in response to receiving confirmation that the anesthesiological procedure is about to begin, start capturing physiological data by means of sensors and data related to the temporal-spatial relationship between the at least one anesthesiologist and the patient and, therefore, to the quality of surveillance provided by the at least one anesthesiologist to patient, via the interaction between the at least one portable device with the at least one computing device and/or the at least one first data acquisition device, and transmitting in real time such collected data from the at least one first data acquisition device and portable device to the at least one computing device and from there to the remote storage where an immutable copy of the collected and preliminary data linked via the at least one generated file is stored;
receiving confirmation from the at least one anesthesiologist, by authentication on the at least one computing device, that the anesthesiological procedure has ended; and
in response to receiving confirmation of completion of the anesthesiological procedure, finishing the transmission and recording of data.

40. The method of claim 39, further comprising the step of carrying out a checklist sub-process consisting of displaying on the computing device a plurality of check indications and requesting a confirmation of compliance from the anesthesiologist.

41. The method of claim 39, further comprising the step of receiving from the anesthesiologist confirmation about geolocation and synchronization with Coordinated Universal Time (UTC) after logging in.

42. The method of claim 39, further comprising the step of receiving supplementary data input during the anesthesiological procedure.

43. The method of claim 39, further comprising the step of stochastically requesting, through an authentication system, the authentication of the at least one anesthesiologist in one or more of: at least one portable device and at least one computing device.

44. The method of claim 39, wherein, when the at least one portable device is a wearable device that is attachable to the body of the anesthesiologist through an opening and closing fastener provided with an electronic contact, the method further comprises the step of requesting, through an authentication system, the authentication of the anesthesiologist on the wearable device in response to detecting the opening and/or closing of said electronic contact.

45. The method of claim 43, wherein, when the at least one portable device is a wearable device that is attachable to the body of the anesthesiologist through an opening and closing fastener provided with an electronic contact, the method further comprises the step of increasing the frequency of stochastic authentication requests for the anesthesiologist on the wearable device in response to repeatedly detecting the opening and/or closing of said electronic contact.

46. The method of claim 39, wherein, when the at least one portable device is a wearable device that comprises at least one motion sensor capable of detecting movement signals from the anesthesiologist, the method further comprises the step of requesting, through an authentication system, the authentication of the anesthesiologist on the wearable device in response to detecting a sudden movement that could indicate that the wearable device was removed from the anesthesiologist's body.

47. The method of claim 43, wherein, when the at least one portable device is a wearable device that comprises at least one motion sensor capable of detecting movement signals from the anesthesiologist, the method further comprises the step of increasing the frequency of stochastic authentication requests for the anesthesiologist on the wearable device in response to repeatedly detecting a sudden movement that could indicate that the wearable device was removed from the anesthesiologist's body.

48. The method of claim 39, wherein, when the at least one portable device comprises at least one motion sensor capable of detecting movement signals from the anesthesiologist, the method further comprises the step of requesting, through an authentication system, the authentication of the anesthesiologist on the portable device in response to detecting an absence of movement or detecting regular movements thereof for a period of time greater than a pre-established time considered prudential.

49. The method of claim 43, wherein, when the at least one portable device comprises at least one motion sensor capable of detecting movement signals from the anesthesiologist, the method further comprises the step of increasing the frequency of stochastic authentication requests for the anesthesiologist on the portable device in response to repeatedly detecting an absence of movement or detecting regular movements thereof for a period of time greater than a pre-established time considered prudential.

50. The method of claim 39, wherein, when the connection of the at least one portable device with the at least one first data acquisition device and/or the at least one computing device is a first wireless connection, the method further comprises the step of requesting, through an authentication system, the authentication of the at least one anesthesiologist in one or more of: at least one portable device and at least one computing device, in response to detecting a weak connection or loss of connection between the at least one portable device and the at least one computing device and/or the at least one first data acquisition device for a period of time greater than a pre-established time considered prudential.

51. The method of claim 43, wherein, when the connection of the at least one portable device with the at least one first data acquisition device and/or the at least one computing device is a first wireless connection, the method further comprises the step of increasing the frequency of stochastic authentication requests for the at least one anesthesiologist in one or more of: at least one portable device and at least one computing device, in response to repeatedly detecting a weak connection or loss of connection between the at least one portable device and the at least one computing device and/or the at least one first data acquisition device for a period of time greater than a pre-established time considered prudential.

52. The method of claim 39, wherein, when the at least one portable device additionally comprises a vibration system and at least one motion sensor capable of detecting movement signals from the anesthesiologist and the connection of the at least one portable device with the at least one first data acquisition device and/or the at least one computing device is a first wireless connection, the method further comprises the step of emitting a vibratory signal via the at least one portable device in response to it detecting the absence of movement or detecting regular movements in it and/or a weak connection or loss of connection with the at least one computing device and/or the at least one first data acquisition device, for a period of time greater than a pre-established period considered prudential.

53. The method of claim 39, wherein, when the at least one portable device further comprises an audible alarm system and at least one motion sensor capable of detecting movement signals from the anesthesiologist and the connection of the at least one portable device with the at least one first data acquisition device and/or the at least one computing device is a first wireless connection, the method further comprises the step of emitting an audible signal via the alarm system of the at least one portable device in response to it detecting the absence of movement or detecting regular movements in it and/or a weak connection or loss of connection between the at least one portable device and the at least one computing device and/or the at least one first data acquisition device, for a period of time greater than a pre-established period considered unsafe for the patient.
